(19) United States
(12) Patent Application Publication
(10) Pub. No.: US 2024/0425439 A1
(43) Pub. Date: Dec. 26, 2024

(54) NEW PROCESS FOR CATALYTIC PREPARATION OF OXALATE AND OXAMIDE COMPOUNDS

(71) Applicant: FAIRBRICS, CLICHY (FR)

(72) Inventors: Ali NASRALLAH, Les Ulis (FR); Tetiana KRACHKO, Massy (FR); Tawfiq NASR ALLAH, Paris (FR)

(73) Assignee: FAIRBRICS, CLICHY (FR)

(21) Appl. No.: 18/690,455

(22) PCT Filed: Sep. 9, 2022
(86) PCT No.: PCT/EP2022/075152

§ 371 Date: Mar. 8, 2024

(30) Foreign Application Priority Data
Sep. 10, 2021 (FR) FR2109523

(51) Int. Cl. C07C 51/145; B01J 31/12; B01J 31/28; C07C 231/02

(52) U.S. Cl. CPC C07C 51/145; B01J 31/12; B01J 31/28; C07C 231/02; C07C 2531/12; C07C 2531/28

(57) ABSTRACT

An M-NHC catalyst, in which M represents Pd or Pt and NHC represents an N-heterocyclic carbene group, including at least one M atom linked to at least one N-heterocyclic carbene ligand, and the method of using the M-NHC catalyst for selectively preparing oxalates or oxamides, from carbon monoxide, an oxidant, in particular molecular oxygen or air, and an alcohol or an amine respectively, optionally in the presence of a promoter.

This invention relates to a new method for preparing oxalate and oxamide compounds by catalytic means.

### CONTEXT OF THE INVENTION

Oxalates and oxamides are important raw materials in the organic chemical industry, used on a large scale to produce various dyes, medicines, important solvents, extraction agents and various intermediates in the fine chemicals industry.

For example, the hydrogenation of oxalates and oxamides can produce ethylene glycol, which is an important raw material in the chemical industry.

A traditional production method for oxalates uses the esterification of oxalic acid with alcohols. This production technique is costly, energy-intensive, polluting and involves unreasonable use of raw materials.

A traditional production method for oxamides is based on the reaction of oxalic acid, or a derivative thereof, with an amine. However, this method has its drawbacks, as it involves the use of expensive or toxic and corrosive reagents. The use of such highly reactive starting compounds easily leads to undesirable side reactions, lowering reaction selectivity and yields.

Oxalate and oxamide compounds can be synthesised directly by oxidative carbonylation of an alcohol or an amine respectively, in the presence of a palladium (Pd) or platinum (Pt) catalyst. A crucial step in the industrial oxidative reaction catalysed by palladium or platinum is the efficient regeneration of the metal atom in the +2 oxidation state from the metal atom in the 0 oxidation state that is reduced in the reaction. In general, it is quite difficult to reoxidise Pd(0) directly to Pd(II) or Pt(0) to Pt(II) using molecular oxygen, so an additional oxidant is usually used.

Pd(0) or Pt(0) can be reoxidised using metal redox couples or benzoquinones. The main disadvantages are:
(1) the use of too many metals, leading to corrosive damage to installations, and
(2) the difficulty of separating the reaction product from the oxidant (for example, when benzoquinone is used as an oxidant, hydroquinone is formed as a by-product).

In addition, prior art methods for the preparation of oxalates are carried out under anhydrous conditions and optionally with a dehydrating agent, since the production of an oxalate is prevented by the water formed in situ when oxygen is used as an oxidant or when water is present in the reagents or solvents. Water deactivates the catalyst.

Another approach to oxalate synthesis is based on the use of alkyl nitrites (RONO), explosive compounds that act as effective reoxidants for Pd(0) and also act as good nucleophiles for CO via their alkoxy function (RO). However, secondary reactions occur, in particular the production of nitric acid.

Prior art methods disclose the use of organometallic complexes of palladium or platinum as homogeneous catalysts for the oxidative carbonylation of alcohols to oxalates and amines to oxamides. Pd(II) salts such as PdCl₂, PdBr₂, Pd(acac)₂, Pd(OAc)₂, PdSO₄, Pd(NO₃)₂ alone or in combination with a phosphine such as Ph₂EtP, PhEt₂P, (PhO)₃P, Ph₃P or with other ligands or Pt(II) salts such as PtCl₂.

However, although the presence of ligands in palladium catalysts has been shown to improve the performance of the catalyst, when operating with strong oxidants such as O₂ and RONO, the phosphorus donor ligands, the P-donors, can be easily oxidised, leading to the degradation of the catalyst into toxic products, which need to be removed by an additional purification step.

For many years, we have been looking for a low-cost, environmentally-friendly way of preparing oxalates and oxamides.

One of the aims of the invention is the use a palladium-(N-heterocyclic carbene) catalyst or a platinum-(N-heterocyclic carbene) catalyst to prepare oxalates or oxamides.

Another aim of the present invention is the use of a palladium-(N-heterocyclic carbene) catalyst or a platinum-(N-heterocyclic carbene) catalyst enabling operation under oxidation conditions with a strong oxidant such as oxygen.

Another aim of the present invention is the use a palladium-(N-heterocyclic carbene) catalyst or a platinum-(N-heterocyclic carbene) catalyst which is stable during the oxidative carbonylation reaction enabling oxalate and oxamide to be prepared and which can be recovered and reused.

Another aim of the invention is the use a palladium-(N-heterocyclic carbene) catalyst or a platinum-(N-heterocyclic carbene) catalyst, advantageously immobilised on a support, to obtain a recyclable, high-performance supported catalyst.

One of the aims of the invention, in the particular case of the preparation of oxalates, concerns the use of a palladium-(N-heterocyclic carbene) catalyst or of a platinum-(N-heterocyclic carbene) catalyst in an oxidative carbonylation method, under particular conditions which may make it possible to avoid the use of:
noxious and toxic reagents such as nitric oxide (NO),
dehydrating agents,
transition metal co-catalysts, or
of P-donor ligands.

Another aim of the invention is the use of a palladium-(N-heterocyclic carbene) catalyst or a platinum-(N-heterocyclic carbene) catalyst in a method for the oxidative carbonylation of an alcohol or an amine leading to an efficient yield.

Another aim of the invention is to use a palladium-(N-heterocyclic carbene) catalyst or a platinum-(N-heterocyclic carbene) catalyst in an oxidative carbonylation method, the selectivity of which is greater than 80% for oxalates or oxamides.

Another aim of the invention is a method for preparing oxalates and oxamides in the presence of a palladium-(N-heterocyclic carbene) catalyst or a platinum-(N-heterocyclic carbene) catalyst.

### Use

A first object of the present invention is the use of an M-NHC catalyst, in which M represents Pd or Pt and NHC represents an N-heterocyclic carbene group, comprising at least one M atom bonded to at least one N-heterocyclic carbene ligand, in the implementation of a method for selectively preparing oxalates or oxamides, from carbon monoxide, an oxidant, in particular molecular oxygen or air, and an alcohol or an amine respectively, optionally in the presence of a promoter.

Within the meaning of this invention, “oxalate” means the dialkyloxalate corresponding to the alcohol used.

By “oxamide” it is meant the 1,1′-oxalyl diamine oxamide derivative corresponding to the amine used.

An M-NHC catalyst is an organometallic complex comprising at least one metal center M consisting of a palladium or platinum atom as the catalytic site, said metal center being bonded to at least one N-heterocyclic carbene ligand.

By “N-heterocyclic carbene” or NHC, it is meant a molecular species with a divalent carbon with 6 valence electrons included in a heterocycle containing at least one nitrogen atom.

By way of non-limiting examples, the following catalysts also fall within the definition according to the invention:
a complex comprising an M center bound to an NHC ligand,
a complex comprising an M center linked to two NHC ligands,
a complex comprising two M centers and two NHC ligands,
a complex comprising two M centers and 4 NHC ligands,
a complex comprising n M centers and n NHC ligands, n being an integer from 1 to 1000, in particular from 1 to 10, and
a complex comprising n M centers and 2n NHC ligands, n being an integer from 1 to 1000, in particular from 1 to 10.

It is clear from the value of the integer n that the catalyst according to the invention can be a polymer.

According to a particular embodiment, the invention concerns the use of an M-NHC catalyst in a method for selectively preparing oxalates or oxamides from carbon monoxide, an oxidant and an alcohol or an amine respectively.

In a particular embodiment, the invention concerns the use of an M-NHC catalyst in a method for selectively preparing oxalates from carbon monoxide, an oxidant and an alcohol.

In a particular embodiment, the invention concerns the use of an M-NHC catalyst in a method for selectively preparing oxamides from carbon monoxide, an oxidant and an amine.

Advantageously, the invention concerns the use of a Pd—NHC palladium catalyst.

According to a particular embodiment, the invention concerns the use of a Pd—NHC catalyst in a method for selectively preparing oxalates or oxamides from carbon monoxide, an oxidant and an alcohol or an amine respectively.

In a particular embodiment, the invention concerns the use of a Pd—NHC catalyst in a method for selectively preparing oxalates from carbon monoxide, an oxidant and an alcohol.

In a particular embodiment, the invention concerns the use of a Pd—NHC catalyst in a method for selectively preparing oxamides from carbon monoxide, an oxidant and an amine.

Advantageously, the invention concerns the use of a Pt—NHC platinum catalyst.

According to a particular embodiment, the invention concerns the use of a Pt—NHC catalyst in a method for selectively preparing oxalates or oxamides from carbon monoxide, an oxidant and an alcohol or an amine respectively.

In a particular embodiment, the invention concerns the use of a Pt—NHC catalyst in a method for selectively preparing oxalates from carbon monoxide, an oxidant and an alcohol.

In a particular embodiment, the invention concerns the use of a Pt—NHC catalyst in a method for selectively preparing oxamides from carbon monoxide, an oxidant and an amine.

According to a particular embodiment, the invention relates to the use as defined above in which the oxidant is chosen from: molecular oxygen (O₂), air, a dione, in particular 1,4-benzoquinone, 1,4-dichloro-2-butene and CuCl₂.

For the purposes of this invention, air is defined as an oxidant. Air is a gas composition comprising, in molar fraction, approximately 78% nitrogen (N₂), 21% oxygen O₂ and approximately less than 1% of other gases including carbon dioxide (CO₂), methane (CH₄) and rare gases including argon, helium, neon, krypton and xenon. Nitrogen is an inert gas, so the oxidising reactivity of air is governed by that of oxygen. Dioxygen is also called molecular oxygen or oxygen in the present invention.

Advantageously, the invention relates to the use as defined above in which molecular oxygen or air is used as the oxidant.

According to a particular embodiment, the invention concerns the use of an M-NHC catalyst in a method for selectively preparing oxalates or oxamides from carbon monoxide, molecular oxygen or air and an alcohol or an amine respectively.

In a particular embodiment, the invention concerns the use of an M-NHC catalyst in a method for selectively preparing oxalates from carbon monoxide, molecular oxygen or air and an alcohol.

In a particular embodiment, the invention concerns the use of an M-NHC catalyst in a method for selectively preparing oxamides from carbon monoxide, molecular oxygen or air and an amine.

Advantageously, the invention relates to the use as defined above of a Pd—NHC catalyst in which molecular oxygen or air is used as the oxidant.

According to a particular embodiment, the invention concerns the use of a Pd—NHC catalyst in a method for selectively preparing oxalates or oxamides from carbon monoxide, molecular oxygen or air and an alcohol or an amine respectively.

In a particular embodiment, the invention concerns the use of a Pd—NHC catalyst in a method for selectively preparing oxalates from carbon monoxide, molecular oxygen or air and an alcohol.

In a particular embodiment, the invention concerns the use of a Pd—NHC catalyst in a method for selectively preparing oxamides from carbon monoxide, molecular oxygen or air and an amine.

Advantageously, the invention relates to the use as defined above of a Pt—NHC catalyst in which molecular oxygen or air is used as the oxidant.

According to a particular embodiment, the invention concerns the use of a Pt—NHC catalyst in a method for selectively preparing oxalates or oxamides from carbon monoxide, molecular oxygen or air and an alcohol or an amine respectively.

In a particular embodiment, the invention concerns the use of a Pt—NHC catalyst in a method for selectively preparing oxalates from carbon monoxide, molecular oxygen or air and an alcohol.

In a particular embodiment, the invention concerns the use of a Pt—NHC catalyst in a method for selectively preparing oxamides from carbon monoxide, molecular oxygen or air and an amine.

Advantageously, the invention relates to the use as defined above of a Pd—NHC catalyst in the presence of a promoter.

According to a particular embodiment, the invention relates to the use of a Pd—NHC catalyst in the implementation of a method for selectively preparing oxalates or oxamides, from carbon monoxide, an oxidant and an alcohol or an amine respectively, in the presence of a promoter.

In a particular embodiment, the invention concerns the use of a Pd—NHC catalyst in a method for selectively preparing oxalates from carbon monoxide, an oxidant and an alcohol in the presence of a promoter.

In a particular embodiment, the invention concerns the use of a Pd—NHC catalyst in a method for selectively preparing oxamides from carbon monoxide, an oxidant and an amine, in the presence of a promoter.

Advantageously, the invention relates to the use as defined above of a Pd—NHC catalyst, in which molecular oxygen or air is used as the oxidant, in the presence of a promoter.

In a particular embodiment, the invention concerns the use of a Pd—NHC catalyst in a method for selectively preparing oxalates or oxamides from carbon monoxide, molecular oxygen or air and an alcohol or an amine respectively, in the presence of a promoter.

In a particular embodiment, the invention concerns the use of a Pd—NHC catalyst in a method for selectively preparing oxalates from carbon monoxide, molecular oxygen or air and an alcohol, in the presence of a promoter.

In a particular embodiment, the invention concerns the use of a Pd—NHC catalyst in a method for selectively preparing oxamides from carbon monoxide, molecular oxygen or air and an amine, in the presence of a promoter.

Advantageously, the catalyst for use according to the invention can be chosen so as not to comprise any phosphorus ligand.

According to a particular embodiment, the invention concerns the use of an M-NHC catalyst which does not have phosphorus ligands, in particular phosphine groups.

Unlike M-phosphine complexes in which the P-donor phosphine ligands oxidise, leading to the formation of degradation products during catalysis, the stability of M-NHC catalysts means that they can be reused and the catalyst is stable in an oxidising reaction environment.

### Supported Catalyst

Advantageously, it is possible to bond the M-NHC catalyst to a support.

According to an advantageous embodiment, the invention relates to the use as defined above of an M-NHC supported catalyst in the implementation of a method for selectively preparing oxalates or oxamides respectively from carbon monoxide, an oxidant and an alcohol or an amine respectively.

According to a particular embodiment, the invention relates to the use as defined above of an M-NHC supported catalyst in the implementation of a method for selectively preparing oxalates from carbon monoxide, an oxidant and an alcohol.

According to a particular embodiment, the invention relates to the use as defined above of an M-NHC supported catalyst in the implementation of a method for selectively preparing oxamides from carbon monoxide, an oxidant and an amine.

By “supported M-NHC catalyst” it is meant that the M-NHC catalyst is bound to a support.

The association between the organometallic complex and the support can be achieved in different ways.

The association between the catalyst and the support can be by chemical bonding, including covalent bonds, ionic bonds and/or intermolecular interactions such as hydrogen bonds.

Preferably, the catalyst is covalently bonded to the support.

In a particular embodiment, the catalyst is bound to the said support by at least one of its ligands, thus allowing access to the metal center.

In a particular embodiment, the catalyst is linked to said support via the N-heterocyclic carbene group.

In one embodiment, the support is an oxide, in particular silica, a polymer, a carbon material such as carbon nanotubes and graphene oxide, or magnetic nanoparticles, preferably a polymer or silica.

In one embodiment, the support is in the form of beads.

By way of non-limiting examples, the support consists of polystyrene beads or silica beads, for example in the form of silica gel.

The use of a supported catalyst has the advantage of facilitating the separation of the catalyst from the reaction medium, making it easy to recover and reuse the catalyst.

The use of a supported catalyst also has the advantage of making it possible to fix the catalyst in the reactor in an enclosure such as a cartridge, when operating under continuous flow, and thus to obtain products leaving the reactor free of catalyst.

### M-NHC Catalyst

Advantageously, by way of non-limitation, the use according to the invention as defined above can be implemented with M-NHC catalysts of specific formulae, defined below.

### M-(N-Heterocyclic Carbene) Catalyst Comprising a Metal Center M Linked to an NHC Ligand.

According to a particular embodiment, the invention relates to the use as defined above, in which the catalyst corresponds to Formula I:

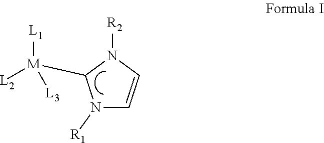

in which:
M represents Pd or Pt,
R₁ and R₂ independently of one another represent a group chosen from:
   C₁ to C₁₀ linear or branched alkyl,
   C₃ to C₁₀ cycloalkyl,
   C₆ to C₂₀ aryl or C₃ to C₂₀ heteroaryl, and
   C₇ to C₂₀ alkyl-aryl or C₄ to C₂₀ alkyl-heteroaryl,
L₁ represents a halogen atom chosen from Cl, Br and I, and
   either L₂ and L₃ are linked and together represent a bidentate ligand,
   or L₂ represents a halogen atom chosen from Cl, Br and I, and L₃ represents a monodentate ligand,
said compound of Formula I can be bonded to a support, in particular a polymer or silica, by at least one of the groups R₁ or R₂.

According to a particular embodiment, the invention relates to the use as defined above, the said compound of Formula I being bonded to a support, in particular a polymer or silica, by at least one of the groups R₁ or R₂.

According to a particular embodiment, the invention relates to the use as defined above, wherein the catalyst is of Formula I, wherein:
L₁ represents an iodine atom, and
   either L₂ and L₃ are linked and together represent a bidentate ligand,
   or L₂ represents a halogen atom chosen from Cl, Br and I and L₃ represents a monodentate ligand.

Surprisingly, the inventors have discovered that using a catalyst carrying an iodinated ligand has the advantage of eliminating the need for a promoter, in particular an iodinated promoter such as tetrabutylammonium iodide (Bu₄NI).

For the purposes of this invention, “C₁ to C₁₀ linear or branched alkyl” refers to an acyclic, saturated, linear or branched carbon chain comprising 1 to 10 carbon atoms. These groups are methyl, ethyl, propyl, butyl, pentyl, hexyl, heptyl, octyl, nonyl and decyl. The definition of propyl, butyl, pentyl, hexyl, heptyl, octyl, nonyl and decyl includes all possible isomers. For example, the term butyl includes n-butyl, iso-butyl, sec-butyl and ter-butyl. One or more hydrogen atoms can be replaced in the alkyl chain.

The term “C₃ to C₁₀ cycloalkyl” refers to: a C₃ cyclopropyl group, a C₄ cyclobutyl group, a C₅ cyclopentyl group, a C₆ cyclohexyl group, a C₇ cycloheptyl group, a C₈ cyclooctyl group, a C₉ cyclononyl group, or a C₁₀ cyclodecyl group, and fused cycloalkane rings such as adamantyl.

The term “C₆ to C₂₀ aryl” refers to an aromatic group comprising 6 to 20 carbon atoms within the aromatic ring, in particular from 6 to 12 carbon atoms, in particular comprising 6, 7, 8, 9, 10, 11, 12, 13, 14, 15, 16, 17, 18, 19 or 20 carbon atoms. The aryl groups according to the present invention can also be substituted, in particular by one or more substituents chosen from a C₁ to C₁₀ linear or branched alkyl group.

Phenyl, toluyl, anisyl and naphthyl o-tolyl, m-tolyl, p-tolyl, o-xylyl, m-xylyl, p-xylyl, are examples of aryl groups.

The term “heteroaryl” refers to an aryl group as defined above, comprising atoms other than carbon atoms, in particular N, O or S within the aromatic ring. Pyridyl, imidazoyl, furfuryl or furanyl are examples of heteroaryl groups according to the present invention.

The term “C₇ to C₂₀ alkyl-aryl” refers to a linear alkyl chain of formula —(CH)₂ₘ—, m varying from 1 to 14, linked to an aryl group as defined above, the alkyl-aryl group consisting of 7 to 20 carbon atoms.

The term “C₄ to C₂₀ alkyl-heteroaryl” refers to a linear alkyl chain of formula —(CH)₂ₘ—, m varying from 1 to 16, linked to a heteroaryl group as defined above, the alkyl-heteroaryl group consisting of 4 to 20 carbon atoms.

Advantageously, the groups R₁ and R₂ can be chosen independently of each other from the following groups: methyl, iso-propyl, tert-butyl, cyclohexyl, adamantyl, mesityl (2,4,6-trimethylphenyl) and diisopropylphenyl.

In one embodiment, the groups R₁ and R₂ are identical. In another embodiment, the groups R₁ and R₂ are different.

By “bidentate ligand” it is meant a charged or neutral molecular group that forms two bonds with the metal center M, in particular through two atoms of the molecular group.

Monodentate ligand” means a charged or neutral molecular group forming a bond with the metal center M, in particular through an atom of the molecular group.

In one embodiment, the ligands L₁, L₂ and L₃ do not contain phosphorus. Advantageously, the catalyst does not contain a phosphine ligand.

The R₁ or R₂ groups can be used to bind the catalyst to a support to form a supported catalyst.

In a particular embodiment, at least one of the R groups₁ and R₂ of the catalyst is bonded to a support, preferably by covalent bonding. The catalyst used is a supported catalyst.

In a particular embodiment, the support is a polymer, in particular polystyrene (PS).

In a particular embodiment, the support is silica, in particular silica gel.

In a particular embodiment, the R₁ and R₂ groups of the catalyst are not bonded to a support.

The catalyst used is a homogeneous catalyst.

### Palladium-(N-Heterocyclic Carbene) Catalyst Comprising a Pd Bound to an NHC Ligand.

According to a particular embodiment, the invention relates to the use as defined above, in which the catalyst corresponds to Formula II

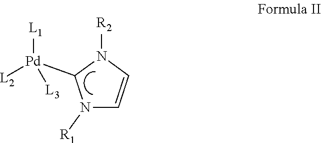

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
L₁ represents a halogen atom chosen from Cl, Br and I, and
   either L₂ and L₃ are linked and together represent a bidentate ligand,
   or L₂ represents a halogen atom chosen from Cl, Br and I, and L₃ represents a monodentate ligand,
said compound of Formula II can be bonded to a support, in particular a polymer or silica, by at least one of the groups R₁ or R₂.

According to a particular embodiment, the invention relates to the use as defined above, the said compound of Formula II can be bonded to a support, in particular a polymer or silica, by at least one of the groups R₁ or R₂.

According to a particular embodiment, the invention relates to the use as defined above, in which the catalyst corresponds to Formula II, in which:
L₁ represents an iodine atom, and
   either L₂ and L₃ are linked and together represent a bidentate ligand,
   or L₂ represents a halogen atom chosen from Cl, Br and I and L₃ represents a monodentate ligand,

According to a particular embodiment, the invention relates to the use as defined above, in which the catalyst corresponds to Formula II, in which:
L₁ and L₂ each represent an iodine atom,
and L₃ represents a monodentate ligand,

According to a particular embodiment, the invention relates to the use as defined above, in which the bidentate ligand is chosen from: acetylacetonate (acac), allyl, cinnamyl and acetate, the bidentate ligand being in particular acetylacetonate.

According to a particular embodiment, the invention relates to the use as defined above, in which the monodentate ligand is chosen from: pyridine, 3-chloropyridine, acetonitrile, triethylamine, or a phosphine-based ligand, in particular triphenylphosphine. Preferably, the monodentate ligand is in particular 3-chloropyridine.

According to a particular embodiment, the invention relates to the use as defined above, in which the monodentate ligand is chosen from: pyridine, 3-chloropyridine, acetonitrile, triethylamine. Preferably the monodentate ligand is 3-chloropyridine.

According to a particular embodiment, the invention relates to the use as defined above, in which the catalyst corresponds to Formula II

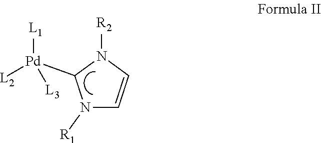

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
L₁ represents a halogen atom chosen from Cl, Br and I, and
   either L₂ and L₃ represent a bidentate ligand, in particular chosen from acetylacetonate (acac), allyl, cinnamyl and acetate, preferably acetylacetonate,
   or L₂ represents a halogen atom chosen from Cl, Br and I,
and L₃ represents a monodentate ligand, in particular selected from: pyridine, 3-chloropyridine, acetonitrile, triethylamine, or a phosphine-based ligand, in particular triphenylphosphine, preferably 3-chloropyridine,
said compound of Formula II can be bonded to a support, in particular a polymer or silica, by at least one of the groups R₁ or R₂.

According to a particular embodiment, the invention relates to the use as defined above, in which the catalyst corresponds to Formula II:

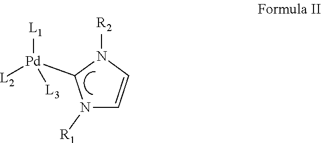

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
L₁ represents an iodine atom, and
   either L₂ and L₃ represent a bidentate ligand, in particular chosen from acetylacetonate (acac), allyl, cinnamyl and acetate, preferably acetylacetonate,
   or L₂ represents a halogen atom chosen from Cl, Br and I and L₃ represents a monodentate ligand, in particular chosen from: pyridine, 3-chloropyridine, acetonitrile, triethylamine, or a phosphine-based ligand, in particular triphenylphosphine, preferably 3-chloropyridine,
said compound of Formula II can be bonded to a support, in particular a polymer or silica, by at least one of the groups R₁ or R₂.

According to a particular embodiment, the invention relates to the use as defined above, in which the catalyst corresponds to Formula II-4

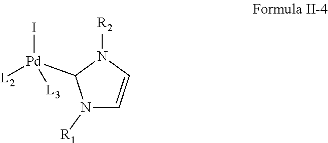

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
in which
   either L₂ and L₃ represent a bidentate ligand, in particular chosen from acetylacetonate (acac), allyl, cinnamyl and acetate, preferably acetylacetonate,
   or L₂ represents a halogen atom chosen from Cl, Br and I and L₃ represents a monodentate ligand, in particular chosen from: pyridine, 3-chloropyridine, acetonitrile, triethylamine, or a phosphine-based ligand, in particular triphenylphosphine, preferably 3-chloropyridine,
said compound of Formula II-4 can be bonded to a support, in particular a polymer or silica, by at least one of the groups R₁ or R₂.

According to a particular embodiment, the invention relates to the use as defined above, in which the catalyst corresponds to Formula II:

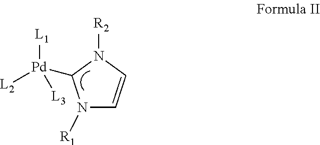

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
L₁ and L₂ each represent an iodine atom, and
L₃ represents a monodentate ligand, in particular selected from: pyridine, 3-chloropyridine, acetonitrile, triethylamine, or a phosphine-based ligand, in particular triphenylphosphine, preferably 3-chloropyridine,
said compound of Formula II can be bonded to a support, in particular a polymer or silica, by at least one of the groups R₁ or R₂.

According to a particular embodiment, the invention relates to the use as defined above, in which the catalyst corresponds to Formula II-5:

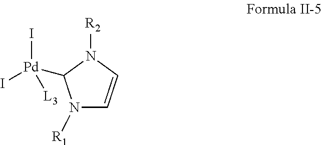

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
L₃ represents a monodentate ligand, in particular selected from: pyridine, 3-chloropyridine, acetonitrile, triethylamine, or a phosphine-based ligand, in particular triphenylphosphine, preferably 3-chloropyridine,
said compound of Formula II-5 can be bonded to a support, in particular a polymer or silica, by at least one of the groups R₁ or R₂.

According to a particular embodiment, the invention relates to the use as defined above, the said compound of Formula II being bonded to a support, in particular a polymer or silica, by at least one of the groups R₁ or R₂.

According to a particular embodiment, the invention relates to the use as defined above, the said compound of Formula II-4 being bonded to a support, in particular a polymer or silica, by at least one of the groups R₁ or R₂.

According to a particular embodiment, the invention relates to the use as defined above, the said compound of Formula II-5 being bonded to a support, in particular a polymer or silica, by at least one of the groups R₁ or R₂.

### Palladium-(N-Heterocyclic Carbene) Catalyst Comprising a Bound Pd and Two NHC Ligands.

In a particular embodiment, the invention relates to the use as defined above, in which the catalyst comprises two palladium-bonded N-heterocyclic carbene groups.

According to a particular embodiment, the invention relates to the use as defined above, in which the catalyst corresponds to Formula III

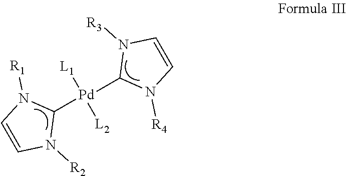

in which:
R₁, R₂, R₃ and R₄ independently of one another represent a group selected from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
or L₁ and L₂ represent independently of each other:
   a halogen atom selected from Cl, Br and I, or
   a monodentate ligand,
or L₁ and L₂ are linked and represent a bidentate ligand,
said compound of Formula III can be bonded to a support, in particular a polymer or silica, by at least one of the groups R₁, R₂, R₃ or R₄,
optionally one of the groups R₁ or R₂ can be linked to R₃ or R₄ and together represent a bidentate group comprising two N-heterocyclic carbene groups.

According to a particular embodiment, the invention relates to the use as defined above, in which the catalyst of Formula III comprises one of the groups R₁ or R₂ linked to R₃ or R₄ and together represent a bidentate group comprising two N-heterocyclic carbene groups.

According to a particular embodiment, the invention relates to the use as defined above, in which the catalyst corresponds to Formula III in which at least one of the groups L₁ and L₂ represents an iodine atom.

According to a particular embodiment, the invention relates to the use as defined above, in which the catalyst corresponds to the following Formula III-2:

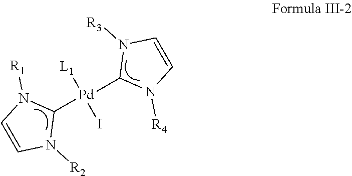

in which:
R₁, R₂, R₃ and R₄ independently of one another represent a group selected from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
in which L₁ represents:
   a halogen atom selected from Cl, Br and I, or
   a monodentate ligand,
said compound of Formula III-2 can be bonded to a support, in particular a polymer or silica, by at least one of the groups R₁, R₂, R₃ or R₄,
optionally one of the groups R₁ or R₂ can be linked to R₃ or R₄ and together represent a bidentate group comprising two N-heterocyclic carbene groups.

According to a particular embodiment, the invention relates to the use as defined above, in which the catalyst corresponds to Formula III in which the groups L₁ and L₂ each represent an iodine atom.

According to a particular embodiment, the invention relates to the use as defined above, in which the catalyst corresponds to the following Formula III-3

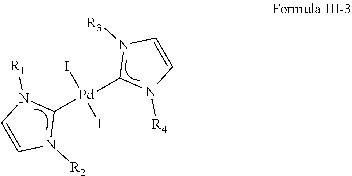

in which:
R₁, R₂, R₃ and R₄ independently of one another represent a group selected from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
said compound of Formula III-3 can be bonded to a support, in particular a polymer or silica, by at least one of the groups R₁, R₂, R₃ or R₄,
optionally one of the groups R₁ or R₂ can be linked to R₃ or R₄ and together represent a bidentate group comprising two N-heterocyclic carbene groups.

According to a particular embodiment, the invention relates to the use as defined above, said compound of Formula III being bonded to a support, in particular a polymer or silica, by at least one of the groups R₁, R₂, R₃ or R₄.

According to a particular embodiment, the invention relates to the use as defined above, the said compound of Formula III-2 being bonded to a support, in particular a polymer or silica, by at least one of the groups R₁, R₂, R₃ or R₄.

According to a particular embodiment, the invention relates to the use as defined above, the said compound of Formula III-3 being bonded to a support, in particular a polymer or silica, by at least one of the groups R₁, R₂, R₃ or R₄.

### Dimer: Palladium-(N-Heterocyclic Carbene) Catalyst Comprising Two Pd and Two NHC Ligands.

According to a particular embodiment, the invention relates to the use as defined above, in which the catalyst corresponds to Formula IV:

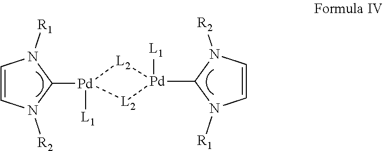

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
L₁ and L₂ represent a halogen atom chosen from Cl, Br and I.

Formula IV shows that the Pd-L₂ bonds are dative and that the complex formed is a dimer with a symmetrical structure.

### Specific Formulae of the Palladium-(N-Heterocyclic Carbene) Catalyst Comprising a Pd Bound to an NHC Ligand.

According to a particular embodiment, the invention relates to the use as defined above, in which the catalyst corresponds to Formula II-1

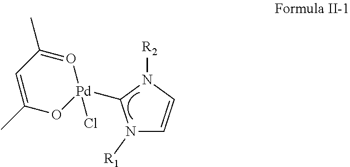

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
said compound of Formula II-1 can be bonded to a support, in particular a polymer or silica, by at least one of the groups R₁ or R₂.

In one embodiment the groups R₁ and R₂ of the catalyst of Formula II-1 are identical. In another embodiment, the groups R₁ and R₂ of the catalyst of Formula II-1 are different.

In one embodiment, the R₁ and R₂ groups of the Formula II-1 catalyst are not bonded to a support.

In another embodiment, at least one of the groups R₁ and R₂ of the catalyst of Formula II-1 is bonded to a support, in particular a polymer or silica.

According to a particular embodiment, the invention relates to the use as defined above, in which the catalyst corresponds to Formula II-2:

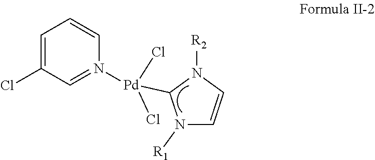

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
said compound of Formula II-2 can be bonded to a support, in particular a polymer or silica, by at least one of the groups R₁ or R₂.

In one embodiment the groups R₁ and R₂ of the catalyst of Formula II-2 are identical. In another embodiment, the groups R₁ and R₂ of the catalyst of Formula II-2 are different.

In one embodiment, the R₁ and R₂ groups of the Formula II-2 catalyst are not bonded to a support.

In another embodiment, at least one of the groups R₁ and R₂ of the catalyst of Formula II-2 is bonded to a support, in particular a polymer or silica.

According to a particular embodiment, the invention relates to the use as defined above, in which the catalyst corresponds to Formula II-3

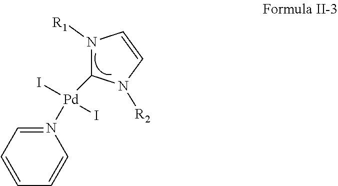

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
said compound of Formula II-3 can be bonded to a support, in particular a polymer or silica, by at least one of the groups R₁ or R₂.

In one embodiment the groups R₁ and R₂ of the catalyst of Formula II-3 are identical. In another embodiment, the groups R₁ and R₂ of the catalyst of Formula II-3 are different.

In one embodiment, the R₁ and R₂ groups of the Formula II-3 catalyst are not bonded to a support.

In another embodiment, at least one of the groups R₁ and R₂ of the catalyst of Formula II-3 is bonded to a support, in particular a polymer or silica.

According to a particular embodiment, the invention relates to the use as defined above, in which the catalyst is bound to polystyrene.

According to a particular embodiment, the invention relates to the use as defined above, wherein the catalyst is selected from:

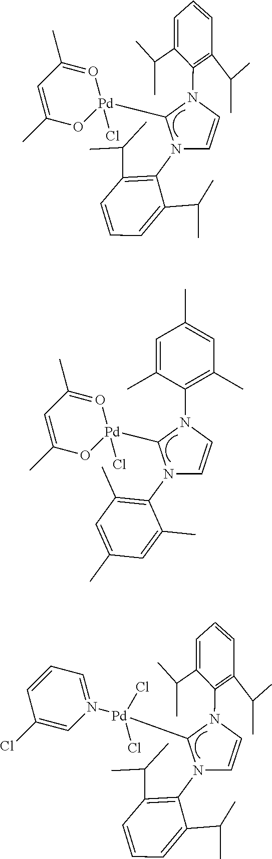

or in which the catalyst bound to a support, is chosen from:

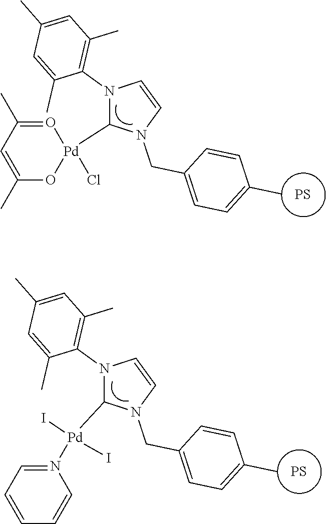

According to a particular embodiment, the invention relates to the use defined above, in which the catalyst corresponds to Formula II-1 or Formula II-2 or Formula II-3

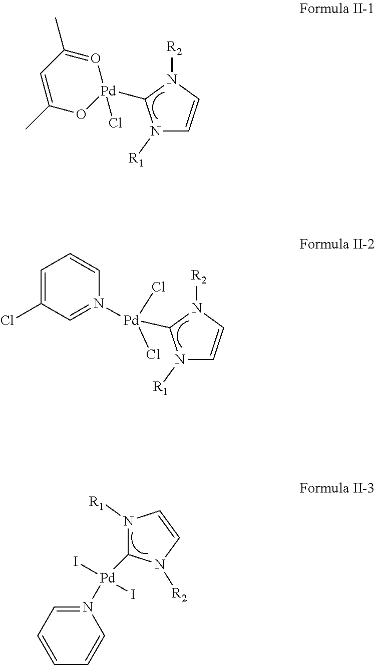

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
said catalyst can be bonded to a support, in particular a polymer or silica, by at least one of the groups R₁ or R₂,
in particular said catalyst is selected from:

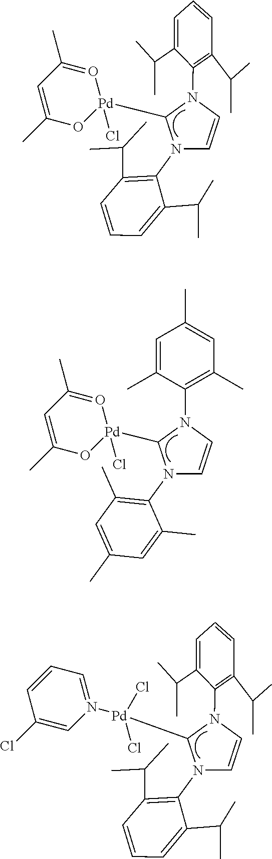

or in which the catalyst, bound to a support (PS), is chosen from:

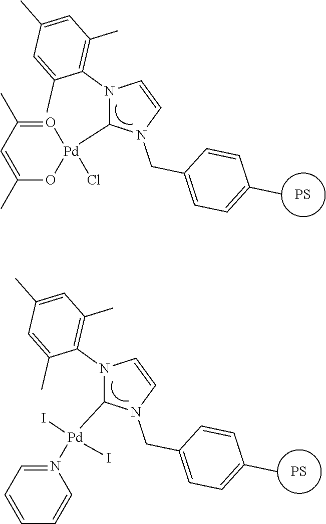

According to a particular embodiment, the invention relates to the use as defined above, in which the catalyst corresponds to Formula III-1

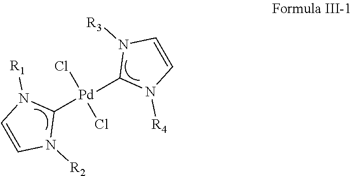

in which:
R₁, R₂, R₃ and R₄ independently of one another represent a group selected from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
said compound of Formula III-1 can be bonded to a support, in particular a polymer or silica, by at least one of the groups R₁, R₂, R₃ or R₄,
optionally one of the groups R₁ or R₂ can be linked to R₃ or R₄ and together represent a bidentate group comprising two N-heterocyclic carbene groups.

In one embodiment, the groups R₁, R₂, R₃ and R₄ of the catalyst of Formula III-1 are not bonded to a support.

According to a particular embodiment, the invention relates to the use as defined above, the said compound of Formula III-1 being bonded to a support, in particular a polymer or silica, by at least one of the groups R₁, R₂, R₃ or R₄.

According to a particular embodiment, the invention relates to the use as defined above, wherein the catalyst of Formula III-1, bound to a support is selected from:

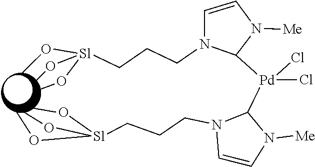

### Platinum-(N-Heterocyclic Carbene) Catalyst Comprising a Pt Bonded to an NHC Ligand.

According to a particular embodiment, the invention relates to the use as defined above, in which the catalyst corresponds to Formula V

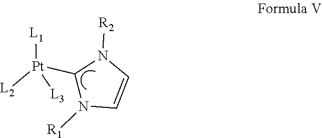

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
L₁ represents a halogen atom chosen from Cl, Br and I, and
either L₂ and L₃ are linked and together represent a bidentate ligand,
   or L₂ represents a halogen atom chosen from Cl, Br and I, and L₃ represents a monodentate ligand,
or L₁, L₂ and L₃ are linked and together represent a tridentate ligand,
said compound of Formula V can be bonded to a support, in particular a polymer or silica, by at least one of the groups R₁ or R₂.

According to a particular embodiment, the invention relates to the use as defined above, in which the bidentate ligand is ethylenediamine.

According to a particular embodiment, the invention relates to the use as defined above, in which the monodentate ligand is chosen from: pyridine, 3-chloropyridine, cyclohexylamine, morpholine and dimethylsulphide, a phosphine in particular triphenylphosphine (PPh₃), ammonia (NH₃) and dimethylsulphoxide (DMSO).

According to a particular embodiment, the invention relates to the use as defined above, in which the tridentate ligand is chosen from terpyridine and diethylenetriamine.

### Platinum-(N-Heterocyclic Carbene) Catalyst Comprising a Pt Bonded to 2 NHC Ligands.

According to a particular embodiment, the invention relates to the use as defined above, in which the catalyst corresponds to Formula VI:

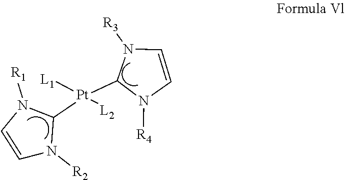

in which:
R₁, R₂, R₃ and R₄ independently of one another represent a group selected from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
or L₁ and L₂ represent independently of each other:
   a halogen atom selected from Cl, Br and I, or
   a monodentate ligand,
or L₁ and L₂ are linked and represent a bidentate ligand,
said compound of Formula VI can be bonded to a support, in particular a polymer or silica, by at least one of the groups R₁, R₂, R₃ or R₄,
optionally one of the groups R₁ or R₂ can be linked to R₃ or R₄ and together represent a bidentate group comprising two N-heterocyclic carbene groups.

### Method

A second object of the present invention is a method for preparing an oxalate compound or an oxamide compound, from carbon monoxide (CO), an oxidant in particular molecular oxygen or air, an alcohol or an amine respectively, catalysed by an M-NHC catalyst, in which M represents the palladium atom (Pd) or the platinum atom (Pt) and NHC represents an N-heterocyclic carbene ligand.

Advantageously, under particular conditions, the presence of a promoter, a base, a solvent or a heating step is optional.

The reaction balance for the oxidative carbonylation of an alcohol or an amine for the method for preparing oxalates or oxamides according to the invention is written as follows

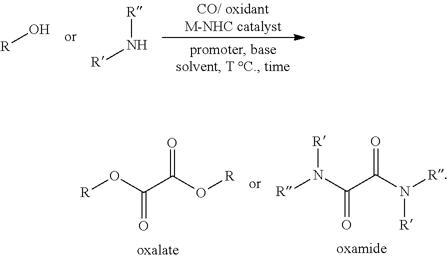

According to a particular embodiment, the invention relates to a method for preparing an oxalate compound or an oxamide compound comprising:
a step A of contacting an alcohol or an amine, respectively, with:
   carbon monoxide,
   an oxidant,
   an M-NHC catalyst, in which M represents Pd or Pt and NHC represents an N-heterocyclic carbene group, comprising at least one M atom linked to at least one N-heterocyclic carbene ligand, and
   optionally a promoter
   optionally a base,
   optionally a solvent,
   to obtain a reaction medium,
optionally a step B of heating the reaction medium,
   to obtain the oxatate compound or the oxamide compound.

According to a particular embodiment, the invention relates to a method as defined above for preparing an oxalate compound comprising:
a step A of contacting an alcohol with:
   carbon monoxide,
   an oxidant,
   an M-NHC catalyst, and
   optionally a promoter,
   optionally a base,
   optionally a solvent,
   to obtain a reaction medium,
optionally a step B of heating said reaction medium,
   to obtain the oxatate compound.

According to a particular embodiment, the invention relates to a method as defined above for preparing an oxamide compound comprising:
a step A of contacting an amine, respectively, with:
   carbon monoxide,
   an oxidant,
   an M-NHC catalyst, and
   optionally a promoter,
   optionally a base,
   optionally a solvent,
   to obtain a reaction medium,
optionally a step B of heating said reaction medium,
   to obtain the oxamide compound.

Advantageously, the invention relates to a method for preparing an oxalate compound or an oxamide compound, using a Pd—NHC palladium catalyst.

According to a particular embodiment, the invention relates to a method as defined above for preparing an oxalate compound or an oxamide compound comprising:
a step A of contacting an alcohol or an amine, respectively, with:
   carbon monoxide,
   an oxidant,
   a Pd—NHC catalyst, and
   optionally a promoter
   optionally a base,
   optionally a solvent,
   to obtain a reaction medium,
optionally a step B of heating said reaction medium,
   to obtain the oxatate compound or the oxamide compound.

According to a particular embodiment, the invention relates to a method as defined above for preparing an oxalate compound comprising:
a step A of contacting an alcohol with:
   carbon monoxide,
   an oxidant,
   a Pd—NHC catalyst, and
   optionally a promoter,
   optionally a base,
   optionally a solvent,
   to obtain a reaction medium,
optionally a step B of heating said reaction medium,
   to obtain the oxatate compound.

According to a particular embodiment, the invention relates to a method as defined above for preparing an oxamide compound comprising:
a step A of contacting an amine, respectively, with:
   carbon monoxide,
   an oxidant,
   a Pd—NHC catalyst, and
   optionally a promoter,
   optionally a base,
   optionally a solvent,
   to obtain a reaction medium,
optionally a step B of heating said reaction medium,
   to obtain the oxamide compound.

Advantageously, the invention relates to a method for preparing an oxalate compound or an oxamide compound, using a Pt—NHC platinum catalyst.

According to a particular embodiment, the invention relates to a method as defined above for preparing an oxalate compound or an oxamide compound comprising:
a step A of contacting an alcohol or an amine, respectively, with:
   carbon monoxide,
   an oxidant,
   a Pt—NHC catalyst, and
   optionally a promoter
   optionally a base,
   optionally a solvent,
   to obtain a reaction medium,
optionally a step B of heating said reaction medium,
   to obtain the oxatate compound or the oxamide compound.

According to a particular embodiment, the invention relates to a method as defined above for preparing an oxalate compound comprising:
a step A of contacting an alcohol with:
   carbon monoxide,
   an oxidant,
   a Pt—NHC catalyst, and
   optionally a promoter,
   optionally a base,
   optionally a solvent,
   to obtain a reaction medium,
optionally a step B of heating said reaction medium,
   to obtain the oxatate compound.

According to a particular embodiment, the invention relates to a method as defined above for preparing an oxamide compound comprising:
a step A of contacting an amine, respectively, with:
   carbon monoxide,
   an oxidant,
   a Pt—NHC catalyst, and
   optionally a promoter,
   optionally a base,
   optionally a solvent,
   to obtain a reaction medium,
optionally a step B of heating said reaction medium,
   to obtain the oxamide compound.

According to a particular embodiment, the invention relates to a method as defined above, of an oxalate compound or an oxamide compound, in which the oxidant is chosen from: molecular oxygen (O₂), air, a dione in particular 1,4-benzoquinone, 1,4-dichloro-2-butene and CuCl₂.

Advantageously, the oxidant is molecular oxygen or air.

According to a particular embodiment, the invention relates to a method as defined above for preparing an oxalate compound or an oxamide compound comprising:
a step A of contacting an alcohol or an amine, respectively, with:
   carbon monoxide,
   oxygen or air,
   an M-NHC catalyst, and
   a promoter
   optionally a base,
   optionally a solvent,
   to obtain a reaction medium,
optionally a step B of heating said reaction medium,
   to obtain the oxatate compound or the oxamide compound.

According to a particular embodiment, the invention relates to a method as defined above for preparing an oxalate compound comprising:
a step A of contacting an alcohol with:
   carbon monoxide,
   oxygen or air,
   an M-NHC catalyst, and
   a promoter,
   optionally a base,
   optionally a solvent,
   to obtain a reaction medium,
optionally a step B of heating the reaction medium,
   to obtain the oxatate compound.

According to a particular embodiment, the invention relates to a method as defined above for preparing an oxamide compound comprising:
a step A of contacting an amine, respectively, with:
   carbon monoxide,
   oxygen or air,
   an M-NHC catalyst, and
   a promoter,
   optionally a base,
   optionally a solvent,
   to obtain a reaction medium,
optionally a step B of heating the reaction medium,
   to obtain the oxamide compound.

Advantageously, the invention relates to a method for preparing an oxalate compound or an oxamide compound, using a Pd—NHC palladium catalyst, in the presence of oxygen or air as oxidant.

According to a particular embodiment, the invention relates to a method as defined above for preparing an oxalate compound or an oxamide compound comprising:
a step A of contacting an alcohol or an amine, respectively, with:
   carbon monoxide,
   oxygen or air,
   a Pd—NHC catalyst, and
   a promoter
   optionally a base,
   optionally a solvent,
   to obtain a reaction medium,
optionally a step B of heating the reaction medium,
   to obtain the oxatate compound or the oxamide compound.

According to a particular embodiment, the invention relates to a method as defined above for preparing an oxalate compound comprising:
a step A of contacting an alcohol with:
   carbon monoxide,
   oxygen or air,
   a Pd—NHC catalyst, and
   a promoter,
   optionally a base,
   optionally a solvent,
   to obtain a reaction medium,
optionally a step B of heating the reaction medium,
   to obtain the oxatate compound.

According to a particular embodiment, the invention relates to a method as defined above for preparing an oxamide compound comprising:
a step A of contacting an amine, respectively, with:
   carbon monoxide,
   oxygen or air,
   a Pd—NHC catalyst, and
   a promoter,
   optionally a base,
   optionally a solvent,
   to obtain a reaction medium,
optionally a step B of heating the reaction medium,
   to obtain the oxamide compound.

Advantageously, the invention relates to a method for preparing an oxalate compound or an oxamide compound, using a Pt—NHC platinum catalyst, in the presence of oxygen or air as oxidant.

According to a particular embodiment, the invention relates to a method as defined above for preparing an oxalate compound or an oxamide compound comprising:
a step A of contacting an alcohol or an amine, respectively, with:
   carbon monoxide,
   oxygen or air,
   a Pt—NHC catalyst, and
   a promoter
   optionally a base,
   optionally a solvent,
   to obtain a reaction medium,
optionally a step B of heating said reaction medium,
   to obtain the oxatate compound or the oxamide compound.

According to a particular embodiment, the invention relates to a method as defined above for preparing an oxalate compound comprising:
a step A of contacting an alcohol with:
   carbon monoxide,
   oxygen or air,
   a Pt—NHC catalyst, and
   a promoter,
   optionally a base,
   optionally a solvent,
   to obtain a reaction medium,
optionally a step B of heating said reaction medium,
   to obtain the oxatate compound.

According to a particular embodiment, the invention relates to a method as defined above for preparing an oxamide compound comprising:
a step A of contacting an amine, respectively, with:
   carbon monoxide,
   oxygen or air,
   a Pt—NHC catalyst, and
   a promoter,
   optionally a base,
   optionally a solvent,
   to obtain a reaction medium,
optionally a step B of heating said reaction medium,
   to obtain the oxamide compound.

By “promoter” we mean a substance that can improve the properties of a catalyst, such as catalytic activity, selectivity, anti-toxicity, stability, lifetime or prevent deactivation of the catalyst.

According to a particular embodiment, the promoter is chosen from iodine derivatives and ammonium salts. The group of iodine derivatives includes I₂, KI, LiI, HI, NaI and tetrabutylammonium iodide (Bu₄NI). Iodine derivatives have the advantage of being soluble in the reaction medium and can therefore advantageously be used in the preparation method without causing excessive formation of solid products. Preferably tetrabutylammonium iodide (Bu₄NI) is used as the promoter, the latter being soluble at 25° C. and not precipitating during the preparation method.

In Pd(II)-catalysed reactions, promoters are known to enable the generated Pd(0) to be reoxidised in-situ to Pd(II) and thus ensure the catalytic cycle. The promoter can also protect the palladium catalyst from the water present in the system, thus preventing deactivation of the catalyst and avoiding the addition of dehydrating agents to the system.

In a particular embodiment, the method according to the invention is carried out under oxidative carbonylation conditions in the presence of oxygen and the promoter acts as an additional oxidant. The promoter can thus promote the oxidative carbonylation method by allowing the M(0) to be oxidised to M(II) in the M-NHC catalyst during the reaction. It is quite difficult to reoxidise Pd(0) directly to Pd(II) using molecular oxygen, so an additional oxidant is generally used.

The term “reaction medium” refers to all the species brought together during a chemical reaction. It includes in particular the reactants in liquid or gaseous form, the catalyst, and optionally a solvent, additives or promoters.

### Method for the Preparation of Oxalates in the Presence of a Pd—NHC Catalyst.

### Heating Stage

According to a particular embodiment, the invention relates to a method as defined above for preparing an oxalate compound comprising:
a step A of contacting an alcohol with:
   carbon monoxide,
   oxygen or air,
   a promoter,
   a Pd—NHC catalyst, and
   optionally a base,
   optionally a solvent,
   to obtain a reaction medium,
a step B for heating said reaction medium,
   to obtain the oxalate compound.

According to a particular embodiment, the invention relates to a method for the preparation, as defined above, of an oxalate compound, in which the heating step B is carried out at a temperature from 25 and 200° C., in particular from 60 to 110° C., preferably about 90° C.

The expression “from 25 to 200° C.” corresponds to the following ranges: from 25 to 40° C.; from 40 to 60° C.; from 60 to 80° C.; from 80 to 100° C.; from 100 to 120° C.; from 120 to 140° C.; from 140 to 160° C.; from 160 to 180° C.; from 180 to 200° C.

The expression “from 60 to 110° C.” corresponds to the following ranges: from 60 to 70° C.; from 70 to 80° C.; from 80 to 90° C.; from 90 to 100° C.; from 100 to 110° C.

According to a particular embodiment, the invention relates to a method for preparing according to the invention defined above an oxalate compound comprising:
a step A of contacting an alcohol with:
   carbon monoxide,
   oxygen or air,
   a promoter,
   a Pd—NHC catalyst, and
   optionally a base,
   optionally a solvent,
   to obtain a reaction medium,
a step B of heating said reaction medium to obtain the oxalate compound, in particular at a temperature of from 25 to 200° C., in particular from 60 to 110° C., preferably about 90° C.

### Base in the Reaction Medium

The oxalate preparation method can be carried out with or without a base in the reaction medium.

### Presence of a Base

According to a particular embodiment, the invention relates to a method as defined above for preparing an oxalate compound comprising:
a step A of contacting an alcohol with:
   carbon monoxide,
   oxygen or air,
   a base,
   a promoter,
   a Pd—NHC catalyst, and
   optionally a solvent,
   to obtain a reaction medium,
a step B of heating said reaction medium to obtain the oxalate compound.

According to a particular embodiment, the invention relates to a method as defined above for preparing an oxalate compound, wherein said reaction medium comprises a base.

The presence of a base, advantageously chosen in the reaction medium, increases the yield of the reaction.

According to a particular embodiment, the invention relates to a method for preparing an oxalate compound as defined above, in which the base is selected from potassium carbonate (K₂CO₃), sodium carbonate (Na₂CO₃), potassium tert-butylate (KOtBu), potassium phosphate (K₃PO₄) and triethylamine (Et₃N).

According to a particular embodiment, the invention relates to a method for preparing an oxalate compound as defined above, in which the base is triethylamine.

Advantageously, the use of triethylamine (Et₃N) makes it easy to evaporate when the product is isolated, and its presence does not lead to problems with recycling the catalyst, such as the solid bases K₂CO₃ and Na₂CO₃, which precipitate at room temperature while the triethylamine remains in the liquid phase.

### No Added Base

Advantageously, the method according to the invention for preparing an oxalate compound can be carried out without using a base.

According to a particular embodiment, the invention relates to a method as defined above for preparing an oxalate compound comprising:
a step A of contacting an alcohol with:
   carbon monoxide,
   oxygen or air,
   a promoter,
   a Pd—NHC catalyst, and
   optionally a solvent,
   to obtain a reaction medium,
a step B of heating said reaction medium to obtain the oxalate compound.

The absence of base in the reaction medium reduces the number of reagents to be introduced into the method and limits the formation of degradation products.

### Solvent in the Reaction Medium

The method according to the invention for preparing oxalates can be carried out with or without a solvent in the reaction medium.

### Presence of a Solvent

According to a particular embodiment, the invention relates to a method as defined above for preparing an oxalate compound, wherein said reaction medium comprises a solvent.

According to a particular embodiment, the invention relates to a method as defined above for preparing an oxalate compound comprising:
a step A of contacting an alcohol with:
   carbon monoxide,
   oxygen or air,
   optionally a base,
   a promoter,
   a Pd—NHC catalyst, and
   a solvent,
   to obtain a reaction medium,
a step B of heating said reaction medium to obtain the oxalate compound.

According to a particular embodiment, the invention relates to a method as defined above for preparing an oxalate compound comprising:
a step A of contacting an alcohol with:
   carbon monoxide,
   oxygen or air,
   a base,
   a promoter,
   a Pd—NHC catalyst, and
   a solvent,
   to obtain a reaction medium,
a step B of heating said reaction medium to obtain the oxalate compound.

According to a particular embodiment, the invention relates to a method as defined above for preparing an oxalate compound comprising:
a step A of contacting an alcohol with:
   carbon monoxide,
   oxygen or air,
   a promoter,
   a Pd—NHC catalyst, and
   a solvent,
   to obtain a reaction medium,
a step B of heating said reaction medium to obtain the oxalate compound.

### No Solvent Added to the Reaction Medium

According to a particular embodiment, the invention relates to a method for preparing an oxalate compound as defined above, in which the method is carried out in the absence of a solvent, the alcohol from which the oxalate is prepared acting as solvent.

The method for preparing an oxalate compound according to the invention can be carried out without using a solvent. This makes it possible to limit the preparation steps and the presence of degradation products to be treated and to limit the solvent treatment steps. For example, the use of alcohol as a reagent and solvent can simplify the recycling of alcohol in the event of incomplete conversion. There is no need to separate solvents such as acetonitrile from the alcohol.

According to a particular embodiment, the invention relates to a method as defined above for preparing an oxalate compound comprising:
a step A of contacting an alcohol with:
   carbon monoxide,
   oxygen or air,
   optionally a base,
   a promoter, and
   a Pd—NHC catalyst,
   to obtain a reaction medium,
a step B of heating said reaction medium to obtain the oxalate compound.

According to a particular embodiment, the invention relates to a method as defined above for preparing an oxalate compound comprising:
a step A of contacting an alcohol with:
   carbon monoxide,
   oxygen or air,
   a base,
   a promoter, and
   a Pd—NHC catalyst,
   to obtain a reaction medium,
a step B of heating said reaction medium to obtain the oxalate compound.

According to a particular embodiment, the invention relates to a method as defined above for preparing an oxalate compound comprising:
a step A of contacting an alcohol with:
   carbon monoxide,
   oxygen or air,
   a promoter, and
   a Pd—NHC catalyst,
   to obtain a reaction medium,
a step B of heating said reaction medium to obtain the oxalate compound.

### Presence of Water

Water formed in situ or present in the reagents can be harmful and in particular deactivate the palladium catalysts in oxidative carbonylation during an oxalate preparation method.

Unlike other catalysts in the prior art such as those described in U.S. Pat. Nos. 3,393,136, 4,005,130, 4,005,129 and 4,005,128, where the presence of water is detrimental during the oxalate preparation method, the use of palladium-(N-heterocyclic carbene) catalysts under the conditions according to the invention makes it possible to dispense with the use of a dehydrating agent or a reactant dehydration step in the preparation of an oxalate compound.

Advantageously, the method according to the invention using the palladium-(N-heterocyclic carbene) catalyst in the presence of oxygen or air and a promoter enables oxalates to be prepared selectively in the presence of water in the reaction medium.

According to a particular embodiment, the invention relates to the use of a palladium-(N-heterocyclic carbene) catalyst in the implementation of a method for selectively preparing oxalates from carbon monoxide, molecular oxygen or air, a promoter and an alcohol, not requiring anhydrous preparation conditions.

### Alcohol as a Substrate

According to a particular embodiment, the invention relates to a method for preparing, as defined above, an oxalate compound of Formula 2, in which step A comprises contacting an alcohol of Formula 1:

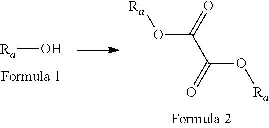

in which Rₐ represents a group chosen from:
C₁ to C₁₀ linear or branched alkyl
C₃ to C₁₀ cycloalkyl

According to a particular embodiment, the invention relates to a method for preparing an oxalate compound as defined above, in which step A comprises contacting an alcohol chosen from methanol, ethanol and isopropanol.

The oxalates prepared with methanol, ethanol and isopropanol are dimethyloxalate, diethyloxalate and diisopropyloxalate respectively, shown below:

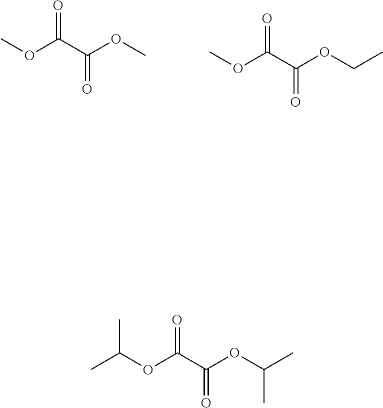

According to a particular embodiment, the invention relates to a method for preparing according to the invention as defined above, an oxalate compound of Formula 2, wherein step A comprises contacting an alcohol of Formula 1

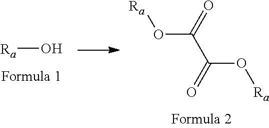

in which Rₐ represents a group chosen from:
C₁ to C₁₀ linear or branched alkyl
C₃ to C₁₀ cycloalkyl
   in particular the alcohol is chosen from methanol, ethanol and isopropanol.

### Method for the Preparation of Oxamides in the Presence of a Pd—NHC Catalyst.

According to a particular embodiment, the invention relates to a method as defined above for preparing an oxamide compound comprising:
a step A of contacting an amine with:
   carbon monoxide,
   oxygen or air,
   optionally a base,
   optionally a promoter,
   a Pd—NHC catalyst, and
   a solvent,
to obtain a reaction medium,
optionally a step B of heating said reaction medium,
   to obtain the oxamide compound.

According to a particular embodiment, the invention relates to a method as defined above for preparing an oxamide compound comprising:
a step A of contacting an amine with:
   carbon monoxide,
   oxygen or air,
   optionally a base,
   a promoter,
   a Pd—NHC catalyst, and
   a solvent,
to obtain a reaction medium,
optionally a step B of heating said reaction medium,
   to obtain the oxamide compound.

According to a particular embodiment, the invention relates to a method as defined above for preparing an oxamide compound comprising:
a step A of contacting an amine with:
   carbon monoxide,
   oxygen or air,
   optionally a base,
   a Pd—NHC catalyst, and
   a solvent,
to obtain a reaction medium,
optionally a step B of heating the reaction medium,
   to obtain the oxamide compound.

### Base in the Reaction Medium

The oxamide preparation method can be carried out with or without a base in the reaction medium.

### No Base Added to the Reaction Medium

According to a particular embodiment, the invention relates to a method as defined above for preparing an oxamide compound, wherein the base is the amine from which said oxamide compound is prepared.

According to a particular embodiment, the invention relates to a method as defined above for preparing an oxamide compound comprising:
a step A of contacting an amine with:
   carbon monoxide,
   oxygen or air,
   a promoter,
   a Pd—NHC catalyst, and
   a solvent,
to obtain a reaction medium,
optionally a step B of heating the reaction medium,
   to obtain the oxamide compound.

### Presence of a Base Added to the Reaction Medium

According to a particular embodiment, the invention relates to a method as defined above for preparing an oxamide compound comprising:
a step A of contacting an amine with:
   carbon monoxide,
   oxygen or air,
   a base,
   a promoter,
   a Pd—NHC catalyst, and
   a solvent,
to obtain a reaction medium,
optionally a step B for heating the reaction medium,
   to obtain the oxamide compound.

According to a particular embodiment, the invention relates to a method for preparing an oxamide compound as defined above, in which the base is chosen from potassium carbonate (K₂CO₃), sodium carbonate (Na₂CO₃), potassium tert-butylate (KOtBu), potassium phosphate (K₃PO₄) and triethylamine (Et₃N).

According to a particular embodiment, the invention relates to a method as defined above for preparing an oxamide compound, wherein said base is an inorganic base, in particular K₂CO₃ According to a particular embodiment, the invention relates to a method as defined above for preparing an oxamide compound, wherein said base is an organic base, in particular triethylamine.

The method for preparing an oxamide compound of the invention can be carried out both with the addition of an inorganic base and with the addition of an organic base. It has been found that the inorganic base K₂CO₃ is as effective as the organic base, triethylamine.

Method with a Heating Step

According to a particular embodiment, the invention relates to a method as defined above for preparing an oxamide compound comprising:
a step A of contacting an amine with:
   carbon monoxide,
   oxygen or air,
   optionally a base,
   a promoter,
   a Pd—NHC catalyst, and
   a solvent,
to obtain a reaction medium,
a step B for heating said reaction medium,
   to obtain the oxamide compound.

According to a particular embodiment, the invention relates to a method as defined above for preparing an oxamide compound comprising:
a step A of contacting an amine with:
   carbon monoxide,
   oxygen or air,
   a base,
   a promoter,
   a Pd—NHC catalyst, and
   a solvent,
to obtain a reaction medium,
a step B for heating said reaction medium,
   to obtain the oxamide compound.

According to a particular embodiment, the invention relates to a method as defined above for preparing an oxamide compound comprising:
a step A of contacting an amine with:
   carbon monoxide,
   oxygen or air,
   a promoter,
   a Pd—NHC catalyst, and
   a solvent,
to obtain a reaction medium,
a step B for heating said reaction medium,
   to obtain the oxamide compound.

According to one embodiment, the method as defined above for preparing an oxamide compound comprises a heating step B at a temperature of 25 to 110° C.

Method without Heating Stage

According to a particular embodiment, the invention relates to a method as defined above for preparing an oxamide compound, in which said reaction medium is maintained at an ambient temperature of 20 to 25° C.

According to a particular embodiment, the invention relates to a method as defined above for preparing an oxamide compound comprising:
a step A of contacting an amine with:
   carbon monoxide,
   oxygen or air,
   optionally a base,
   a promoter,
   a Pd—NHC catalyst, and
   a solvent,
      to obtain a reaction medium comprising the oxamide compound.

According to a particular embodiment, the invention relates to a method as defined above for preparing an oxamide compound comprising:
a step A of contacting an amine with:
   carbon monoxide,
   oxygen or air,
   a base,
   a promoter,
   a Pd—NHC catalyst, and
   a solvent,
      to obtain a reaction medium comprising the oxamide compound.

According to a particular embodiment, the invention relates to a method as defined above for preparing an oxamide compound comprising:
a step A of contacting an amine with:
   carbon monoxide,
   oxygen or air,
   a promoter,
   a Pd—NHC catalyst, and
   a solvent,
      to obtain a reaction medium comprising the oxamide compound.

The method for preparing oxamides according to the invention by oxidative carbonylation can advantageously be carried out at room temperature from 20 to 25° C.

It was observed that the method for preparing oxamides according to the invention in the presence of a base and solvent enabled oxamides to be obtained in the reaction medium without a heating step.

As a result, heating the reaction medium is optional, which represents an industrial advantage in terms of cost and safety.

According to a particular embodiment, the invention relates to a method as defined above for preparing an oxamide compound comprising:
a step A of contacting an amine with:
   carbon monoxide,
   oxygen or air,
   a promoter,
   a Pd—NHC type catalyst, and
   a solvent,
   optionally a base, in particular selected from K₂CO₃ or Et N₃
      to obtain a reaction medium comprising the oxamide.

### The Amine Used as a Substrate

According to a particular embodiment, the invention relates to a method for preparing, as defined above, an oxamide compound of Formula 4 in which step A comprises contacting an amine of Formula 3

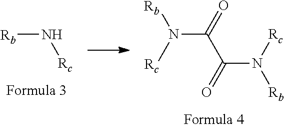

in which R_{b} and R_{c} independently of one another represent:
a hydrogen atom,
C₁ to C₂₀ linear or branched alkyl group
C₂ to C₂₀ linear or branched alkenyl group
C₁ to C₂₀ linear or branched heteroalkyl group, the heteroatom being in particular O or N,
   at least one of the groups R_{b} or R_{c} being other than hydrogen,
   R_{b} and R_{c} can form a cycle.

The term “linear or branched heteroalkyl” refers to an alkyl group, linear or branched, as defined above, comprising atoms other than carbon atoms, in particular N, O or S within the alkyl chain.

The term “linear or branched alkenyl” refers to a linear or branched alkyl group as defined above with a C═C double bond.

According to a particular embodiment, the invention relates to a method for preparing an oxamide compound as defined above, in which step A comprises contacting an amine chosen from: diethylamine, piperidine, pyrrolidine and morpholine.

The oxamides prepared with diethylamine, piperidine, pyrrolidine and morpholine are shown below:

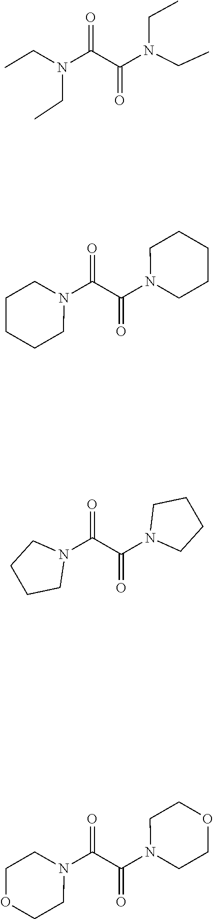 [SMILES: CCN(CC)C(=O)C(=O)N(CC)CC ; O=C(C(=O)N1CCCCC1)N1CCCCC1 ; O=C(C(=O)N1CCCC1)N1CCCC1 ; O=C(C(=O)N1CCOCC1)N1CCOCC1]

According to a particular embodiment, the invention relates to a method for preparing, as defined above, an oxamide compound of Formula 4, in which the groups R_{b} and R_{c} are linked and form a ring.

According to a particular embodiment, the invention relates to a method for preparing, as defined above, an oxamide compound of Formula 4, in which step A comprises contacting an amine of Formula 3:

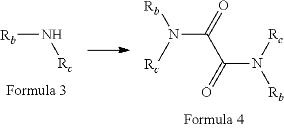

in which R_{b} and R_{c} independently of one another represent:
a hydrogen atom,
C₁ to C₂₀ linear or branched alkyl group
C₂ to C₂₀ linear or branched alkenyl group
C₁ to C₂₀ linear or branched heteroalkyl group, the heteroatom being in particular O or N,
   at least one of the groups R_{b} or R_{c} being other than hydrogen,
   R_{b} and R_{c} can form a cycle,
   in particular the amine is chosen from: diethylamine, piperidine, morpholine, pyrrolidine.

### Common Features of the Method for Preparing Oxalates and Oxamides.

### Catalysts

According to a particular embodiment, the invention relates to a preparation method as defined above, in which the catalyst corresponds to Formula II-B:

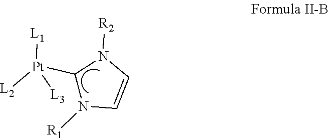

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
L¹ represents a halogen atom chosen from Cl, Br and I, and
   either L² and L³ represent a bidentate ligand,
   or L² represents a halogen atom chosen from Cl, Br and I, and L³ represents a monodentate ligand,
said compound of Formula II-B can be bonded to a support, in particular a polymer or silica, by at least one of the groups R₁ or R₂.

According to a particular embodiment, the invention relates to the preparation method as defined above, in which the catalyst corresponds to Formula II-B, in which:
L₁ represents an iodine atom, and
   either L₂ and L₃ are linked and together represent a bidentate ligand,
   or L₂ represents a halogen atom chosen from Cl, Br and I and L₃ represents a monodentate ligand,

According to a particular embodiment, the invention relates to the preparation method as defined above, in which the catalyst corresponds to Formula II-4B

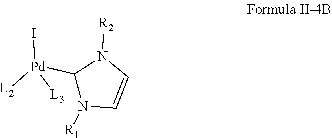

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
in which
   either L₂ and L₃ represent a bidentate ligand, in particular chosen from acetylacetonate (acac), allyl, cinnamyl and acetate, preferably acetylacetonate,
   or L₂ represents a halogen atom chosen from Cl, Br and I and L₃ represents a monodentate ligand, in particular chosen from: pyridine, 3-chloropyridine, acetonitrile, triethylamine, or a phosphine-based ligand, in particular triphenylphosphine, preferably 3-chloropyridine,
said compound of Formula II-4B can be bonded to a support, in particular a polymer or silica, by at least one of the groups R₁ or R₂.

According to a particular embodiment, the invention relates to the preparation method as defined above, in which the catalyst corresponds to Formula II-B, in which:
L₁ and L₂ each represent an iodine atom, and L₃ represents a monodentate ligand.

According to a particular embodiment, the invention relates to the preparation method as defined above, in which the catalyst corresponds to Formula II-5B

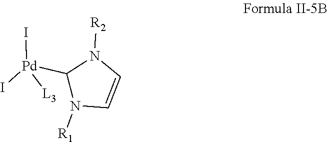

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
L₃ represents a monodentate ligand, in particular selected from: pyridine, 3-chloropyridine, acetonitrile, triethylamine, or a phosphine-based ligand, in particular triphenylphosphine, preferably 3-chloropyridine,
said compound of Formula II-5B can be bonded to a support, in particular a polymer or silica, by at least one of the groups R₁ or R₂.

According to a particular embodiment, the invention relates to a preparation method as defined above, in which the bidentate ligand is chosen from: acetylacetonate (acac), allyl, cinnamyl and acetate, the bidentate ligand being in particular acetylacetonate.

According to a particular embodiment, the invention relates to a preparation method as defined above, in which the monodentate ligand is chosen from: pyridine, 3-chloropyridine, acetonitrile, triethylamine, or a phosphine-based ligand, in particular triphenylphosphine. Preferably the monodentate ligand is in particular 3-chloropyridine.

According to a particular embodiment, the invention relates to a preparation method as defined above, in which the monodentate ligand is chosen from: pyridine, 3-chloropyridine, acetonitrile, triethylamine, preferably the monodentate ligand being in particular 3-chloropyridine.

According to a particular embodiment, the invention relates to the preparation method as defined above, said compound of Formula II-B not being bound to a support.

According to a particular embodiment, the invention relates to the preparation method as defined above, the said compound of Formula II-B being bonded to a support, in particular a polymer or silica, by at least one of the groups R₁ or R₂.

According to a particular embodiment, the invention relates to the preparation method as defined above, the said compound of Formula II-4B being bonded to a support, in particular a polymer or silica, by at least one of the groups R₁ or R₂.

According to a particular embodiment, the invention relates to the preparation method as defined above, the said compound of Formula II-5B being bonded to a support, in particular a polymer or silica, by at least one of the groups R₁ or R₂.

According to a particular embodiment, the invention relates to a preparation method as defined above, in which the catalyst corresponds to Formula II-1B:

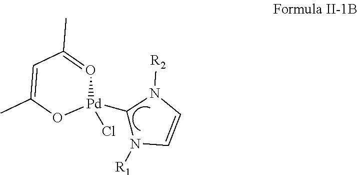

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
said compound of Formula II-1B can be bonded to a support, in particular a polymer or silica, by at least one of the groups R₁ or R₂.

According to a particular embodiment, the invention relates to a preparation method as defined above, in which the catalyst corresponds to Formula II-2B:

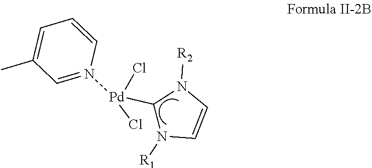

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
said compound of Formula II-2B can be bonded to a support, in particular a polymer or silica, by at least one of the groups R₁ or R₂.

According to a particular embodiment, the invention relates to a preparation method as defined above, in which the catalyst corresponds to Formula II-3B:

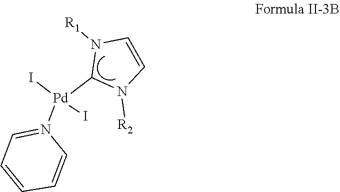

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
said compound of Formula II-3B can be bonded to a support, in particular a polymer or silica, by at least one of the groups R₁ or R₂.

According to a particular embodiment, the invention relates to the preparation method as defined above, the said compound of Formula II-3B being bonded to a support, in particular a polymer or silica, by at least one of the groups R₁ or R₂.

According to a particular embodiment, the invention relates to a preparation method as defined above, in which the support is polystyrene or silica gel, said catalyst being bonded to said polystyrene or silica gel.

According to a particular embodiment, the invention relates to a preparation method as defined above, in which the catalyst is chosen from:

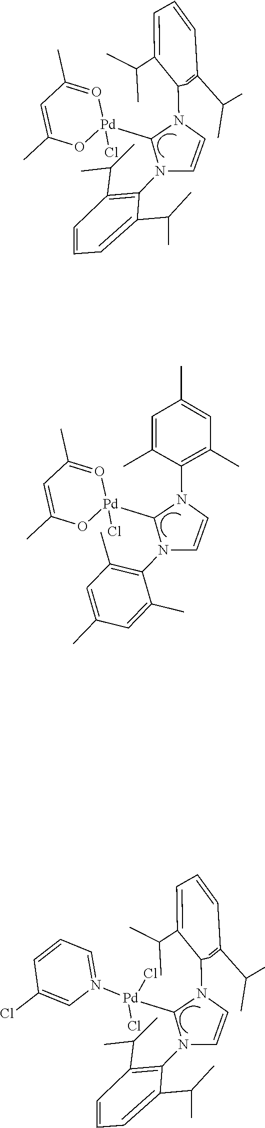

or in which the supported catalyst is selected from:

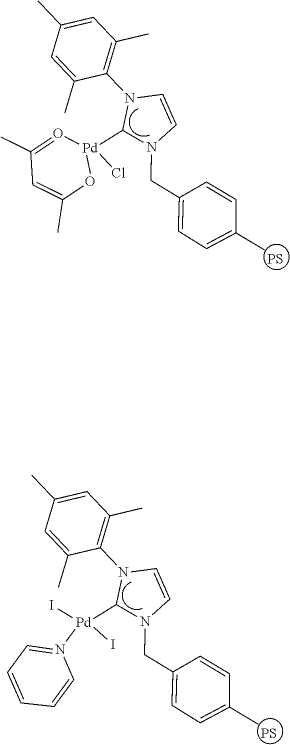

According to a particular embodiment, the invention relates to a preparation method as defined above, in which the catalyst corresponds to Formula III-B:

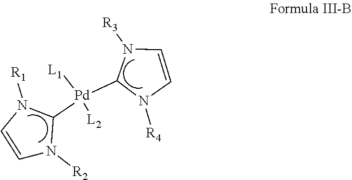

in which:
R₁, R₂, R₃ and R₄ independently of one another represent a group selected from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
either L₁ and L₂ represent independently of each other:
   a halogen atom selected from Cl, Br and I, or
   a monodentate ligand,
or L₁ and L₂ are linked and represent a bidentate ligand, said compound of Formula III-B can be linked to a support, in particular a polymer, by at least one of the groups R₁, R₂, R₃ or R₄,
optionally one of the groups R₁ or R₂ can be linked to R₃ or R₄ and together represent a bidentate group comprising two N-heterocyclic carbene groups.

According to a particular embodiment, the invention relates to the preparation method as defined above, the said compound of Formula III-B being bonded to a support, in particular a polymer or silica, by at least one of the groups R₁, R₂, R₃ or R₄.

According to a particular embodiment, the invention relates to the use as defined above, in which the catalyst corresponds to Formula III-B in which at least one of the groups L₁ and L₂ represents an iodine atom.

According to a particular embodiment, the invention relates to the preparation method as defined above, in which the catalyst corresponds to Formula III-1B

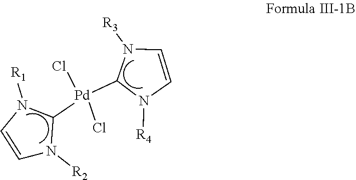

in which:
R₁, R₂, R₃ and R₄ independently of one another represent a group selected from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
said compound of Formula III-1B can be bonded to a support, in particular a polymer or silica, by at least one of the groups R₁, R₂, R₃ or R₄,
optionally one of the groups R₁ or R₂ can be linked to R₃ or R₄ and together represent a bidentate group comprising two N-heterocyclic carbene groups.

According to a particular embodiment, the invention relates to a preparation method as defined above, in which the catalyst of Formula III-1B bound to a support is chosen from:

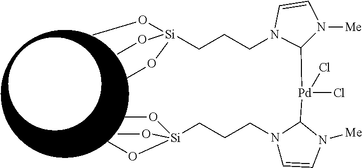

According to a particular embodiment, the invention relates to the preparation method as defined above, in which the catalyst corresponds to Formula III-2B

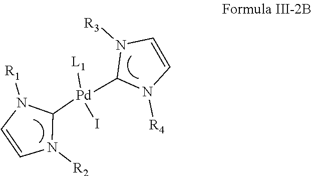

in which:
R₁, R₂, R₃ and R₄ independently of one another represent a group selected from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
in which L₁ represents:
   a halogen atom selected from Cl, Br and I, or
   a monodentate ligand,
said compound of Formula III-2B can be bonded to a support, in particular a polymer or silica, by at least one of the groups R₁, R₂, R₃ or R₄,
optionally one of the groups R₁ or R₂ can be linked to R₃ or R₄ and together represent a bidentate group comprising two N-heterocyclic carbene groups.

According to a particular embodiment, the invention relates to the preparation method as defined above, in which the catalyst corresponds to Formula III-3B

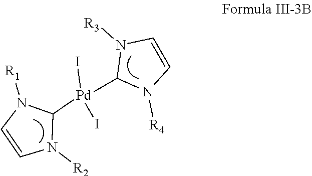

in which:
R₁, R₂, R₃ and R₄ independently of one another represent a group selected from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
said compound of Formula III-3B can be bonded to a support, in particular a polymer or silica, by at least one of the groups R₁, R₂, R₃ or R₄,
optionally one of the groups R₁ or R₂ can be linked to R₃ or R₄ and together represent a bidentate group comprising two N-heterocyclic carbene groups.

According to a particular embodiment, the invention relates to a preparation method as defined above, in which the catalyst corresponds to Formula IV-B:

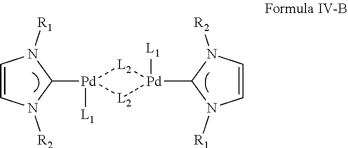

in which:
R₁ and R₂ independently of one another represent a group chosen from:
   C₁ to C₁₀ linear or branched alkyl
   C₃ to C₁₀ cycloalkyl
   C₆ to C₂₀ aryl or C₃ to C₂₀ heteroaryl, and
   C₇ to C₂₀ alkyl-aryl or C₄ to C₂₀ alkyl-heteroaryl,
L¹ and L² represent a halogen atom chosen from Cl, Br and I.

### Other Parameters

According to a particular embodiment, the invention concerns a preparation method as defined above, in which the promoter is chosen from: tetrabutylammonium iodide (Bu₄NI), sodium iodide (NaI) and potassium iodide (KI),
the promoter being in particular tetrabutylammonium iodide.

According to a particular embodiment, the invention relates to a preparation method as defined above, in which the solvent is chosen from: acetonitrile, toluene, 1,4-dioxane, tetrahydrofuran, ethanol, methanol and ethyl acetate,
the solvent being in particular acetonitrile or tetrahydrofuran.

According to a particular embodiment, the invention relates to a preparation method as defined above, in which the oxidant is oxygen or air, the oxygen being used at a rate of 0.5 to 2.5 MPa (5 to 25 bars).

Advantageously, oxygen is used at a rate of 1.5 MPa for the oxalate preparation method.

Advantageously, oxygen is used at a rate of 1.0 MPa for the oxamide preparation method.

The expression MPa corresponds to 10⁶ Pascal and is equivalent to 10 bars.

The expression “from 0.5 to 2.5 MPa” corresponds to the following ranges: from 0.5 to 1.0 MPa; from 1.0 to 1.5 MPa; from 1.5 to 2.0 MPa; from 2.0 to 2.5 MPa.

According to a particular embodiment, the invention relates to a preparation method as defined above, in which carbon monoxide is used from 1.0 to 10.0 MPa (10 to 100 bars), in particular at 6.5 MPa (65 bars).

The expression “from 1.0 to 10.0 MPa” corresponds to the ranges: from 1.0 to 1.5 MPa; from 1.5 to 2.0 MPa; from 2.0 to 2.5 MPa; from 2.5 to 3.0 MPa; from 3.0 to 3.5 MPa; from 3.5 to 4.0 MPa; from 4.0 to 4.5 MPa; from 4.5 to 5.0 MPa; from 5.0 to 5.5 MPa; from 5.5 to 6.0 MPa; from 6.0 to 6.5 MPa; from 6.5 to 7.0 MPa; from 7.0 to 7.5 MPa; from 7.5 to 8.0 MPa; from 8.0 to 8.5 MPa; from 8.5 to 9.0 MPa; from 9.0 to 9.5 MPa; from 9.5 to 10 MPa.

According to a particular embodiment, the invention relates to a preparation method as defined above, in which the carbon monoxide/oxygen pressure ratio used is from 3 to 10, in particular about 4.

The expression “from 3 to 10” corresponds to the ranges: from 3 to 4; from 4 to 5; from 5 to 6; from 6 to 7; from 7 to 8; from 8 to 9; from 9 to 10.

Advantageously, the CO/O₂ pressure ratio is about 4 for the oxalate preparation method.

Advantageously, the CO/O₂ pressure ratio is about 6.5 for the oxamide preparation method.

According to a particular embodiment, the invention relates to a preparation method as defined above, in which the catalyst is used in an amount of 0.001 to 10 mol % relative to the alcohol or amine.

The expression “from 0.001 to 10%” corresponds to the ranges: from 0.001 to 0.005%; from 0.005 to 0.01%; from 0.01 to 0.05%; from 0.05 to 0.1%; from 0.1 to 0.15%; from 0.15 to 0.2%; from 0.2 to 0.5%; from 0.5 to 1%; from 1 to 2%; from 2 to 3%; from 3 to 4%; from 4 to 5%; from 5 to 6%; from 6 to 7%; from 7 to 8%; from 8 to 9%; from 9 to 10%.

According to a particular embodiment, the invention relates to a preparation method as defined above, in which the promoter is used in an amount from 2 to 100 molar equivalents relative to the catalyst.

Advantageously, the promoter is used in a proportion of 5 molar equivalents relative to the catalyst for the oxalate preparation method.

Advantageously, the promoter is used in a proportion of 62.5 molar equivalents relative to the catalyst for the oxamide preparation method.

According to a particular embodiment, the invention relates to a preparation method as defined above, in which the base is used in an amount from 2 to 150 molar equivalents relative to the catalyst.

Advantageously, the base is used in a proportion of 5 molar equivalents relative to the catalyst for the oxalate preparation method.

Advantageously, the base is used in a proportion of 125 molar equivalents relative to the catalyst for the oxamide preparation method.

According to a particular embodiment, the invention relates to a method for preparing, as defined above, an oxalate compound or an oxamide compound comprising:
a step A of contacting an alcohol or an amine, respectively, with:
   carbon monoxide, in particular used from 1.0 to 10.0 MPa, in particular 6.5 MPa,
   an oxidant, in particular oxygen or air, preferably oxygen used from 0.5 to 2.5 MPa,
   an M-NHC catalyst, in which M represents Pd or Pt and NHC represents an N-heterocyclic carbene group, comprising at least one M atom linked to at least one N-heterocyclic carbene ligand, and
   optionally a promoter, in particular selected from: tetrabutylammonium iodide (Bu₄NI), sodium iodide (NaI) and potassium iodide (KI), preferably tetrabutylammonium iodide,
   optionally a base, in particular selected from potassium carbonate (K₂CO₃), sodium carbonate (Na₂CO₃), potassium tert-butylate (KOtBu), potassium phosphate (K₃PO₄) and triethylamine (Et₃N),
   optionally a solvent, in particular acetonitrile, toluene, 1,4-dioxane, tetrahydrofuran, ethanol, methanol and ethyl acetate, preferably acetonitrile and tetrahydrofuran,
   to obtain a reaction medium,
optionally a step B of heating the reaction medium,
   to obtain the oxalate compound or the oxamide compound.

According to a particular embodiment, the invention relates to a preparation method as defined above, in which the method further comprises, after step B of heating, a step C of filtering the reaction medium to obtain a recovered catalyst, and a catalyst-free filtrate, said catalyst being a supported catalyst as defined above.

According to a particular embodiment, the invention relates to a preparation method as defined above, in which the catalyst used in the contacting step A is a catalyst recovered at the end of a filtration step C.

Advantageously, the catalyst recovered after the method of the invention is not degraded and is stable, and it can be reused in another catalytic reaction method. It is therefore possible to repeat the method according to the invention with the same recovered catalyst.

According to a particular embodiment, the invention relates to a method for preparing, as defined above, an oxalate compound or an oxamide compound, in which the catalyst is stable at the end of the reaction and can be reused in another catalytic reaction method.

According to a particular embodiment, the invention relates to a preparation method as defined above, in which steps A, B and C are repeated at least 5 times, without substantial loss of the catalytic activity of the catalyst.

According to a particular embodiment, the invention relates to a preparation method as defined above, in which the method is carried out in continuous flow, the catalyst being a supported catalyst as defined above.

According to a particular embodiment, the invention concerns a continuous flow process for preparing oxalates or oxamides as defined above, in which the catalyst is a supported catalyst introduced into a column or cartridge, or in which the catalyst is suspended in the reaction mixture.

By way of a non-limiting example, the continuous flow process is carried out in a reactor of the following type:
a continuously stirred tank reactor (CSTR),
a flow reactor or a tubular reactor,
a fixed or packed bed reactor.

By way of example, the method according to the invention can be implemented in a flow chemistry apparatus, for example in commercial reactors such as “H-Cube Pro®” or “Phoenix®” from ThalesNano INC. (7 Zahony Street, Graphisoft Park, Building D, H-1031 Budapest, Hungary) or such as the “E-Series” or “R-Series flow chemistry systems” from Vapourtec Ltd (Unit 21/Park Farm Business Center/Fornham Pk, Bury Saint Edmunds IP28 6TS, United Kingdom).

Advantageously, the continuous flow process is carried out at a temperature from 25° C. to 200° C.

Advantageously, the continuous flow process is carried out at a pressure from 0.1 MPa to 4 MPa.

The expression “from 0.1 to 4 MPa” corresponds to the following ranges: from 0.1 to 0.5 MPa; from 0.5 to 1.0 MPa; from 1.0 to 1.5 MPa; from 1.5 to 2.0 MPa; from 2.0 to 2.5 MPa; from 2.5 to 3.0 MPa; 3.0 to 3.5 MPa; from 3.5 to 4.0 MPa.

In a particular embodiment, the continuous flow method is carried out in a reactor in which the gases represent from 10 to 90% of the reactor volume.

The expression “from 10 to 90%” corresponds to the following ranges: from 10 to 20%; from 20 to 30%; from 30 to 40%; from 40 to 50%; from 50 to 60%; from 60 to 70%; from 70 to 80%; from 80 to 90%.

According to a particular embodiment, the continuous flow process is carried out by means allowing a contact time between the reagents of 1 second to 2 hours, in particular 1 second to 2 minutes.

The expression “from 1 second to 2 hours” corresponds to the following ranges: from 1 to 15 seconds; from 15 to 30 seconds; from 30 seconds to 1 minute; from 1 to 2 minutes; from 2 to 15 minutes; from 15 to 30 minutes; from 30 minutes to 1 hour; from 1 to 2 hours.

In a particular embodiment, the continuous flow process comprises means for introducing into the reactor the stream of CO in contact with the substrate (the alcohol or the amine) and the stream of oxygen or air individually or as a mixture.

According to a particular embodiment, the invention relates to a preparation method as defined above, in which the oxalate product, or the oxamide product, is isolated in the absence of carbonate products or urea products respectively, the oxalate/carbonate ratio being greater than 98%, the oxamide/urea ratio being greater than 98%.

According to a particular embodiment, the invention concerns a preparation method as defined above, in which the oxalate product is isolated in the absence of carbonate products, the oxalate/carbonate ratio being greater than 98%.

According to a particular embodiment, the invention concerns a preparation method as defined above, in which the oxamide product is isolated in the absence of urea products, the oxamide/urea ratio being greater than 98%.

By way of non-limiting examples, the oxalate product or the oxamide product can be isolated by distillation or by extraction and recrystallisation.

According to a particular embodiment, the invention relates to a method for preparing, as defined above, an oxalate compound or an oxamide compound, in which the yield obtained is greater than 100 mmol of the oxalate compound or of the oxamide compound per mmol of Pd.

According to a particular embodiment, the invention relates to a method for preparing an oxalate compound as defined above, in which the yield obtained is greater than 100 mmol of the oxalate compound per mmol of Pd.

According to a particular embodiment, the invention relates to a method for preparing an oxamide compound as defined above, in which the yield obtained is greater than 200 mmol of the oxamide compound per mmol of Pd.

Advantageously, the method for preparing oxamides according to the invention makes it possible to achieve yields of more than 90%, in particular of the order of 97%.

The yields of the method for preparing oxalates or oxamides according to the invention can be described in terms of “Number of Catalytic Cycles (NCC)”.

In particular, the catalytic activity under given conditions of temperature, pressure and concentrations of solutes and time of the method for preparing oxalates or oxamides according to the invention can be described in terms of NCC.

The “Number of Catalytic Cycles (NCC)” is defined as the ratio between the number of moles of product formed (n_{prod}) and the number of moles of metal (Pd or Pt) in the catalyst (n_{cat}):

$$NCC = \frac{{n}_{prod}}{{n}_{cat}}$$

Unlike the Turnover Number (TON), which represents the maximum number of catalytic cycles that a catalyst can achieve before its total and irreversible degradation, the Catalytic Cycle Number represents the total number of catalytic cycles achieved by the catalyst under given reaction conditions. At the end of the reaction, the catalyst used would not necessarily be degraded and could therefore be reused. The NCC is therefore not a measure of the lifetime of a catalyst, but a measure of the productivity of the catalyst under the given conditions of the catalysed reaction.

According to a particular embodiment, the invention relates to a method for preparing an oxalate compound as defined above, in which the NCC obtained is greater than 100.

According to a particular embodiment, the invention relates to a method for preparing an oxamide compound as defined above, in which the NCC is greater than 200.

According to a particular embodiment, the invention relates to a method for preparing, as defined above, an oxalate compound or an oxamide compound, in which the selectivity towards the oxalate product, or the oxamide product, is greater than 70%, in particular about 80%.

According to a particular embodiment, the invention relates to a method for preparing, as defined above, an oxalate compound in which the selectivity towards the oxalate product is greater than 75%, in particular about 80%.

According to a particular embodiment, the invention relates to a method for preparing an oxamide compound as defined above, in which the selectivity towards the oxamide product is greater than 95%, in particular about 98%.

The following examples illustrate the invention, without limiting its scope.

### EXAMPLES

### Example 1: Catalysts

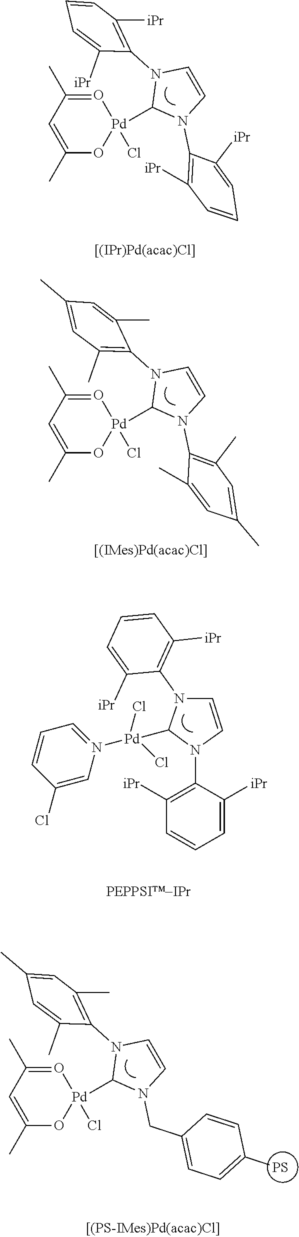

The PEPPSI™—IPr catalyst can be commercially obtained from Sigma Aldrich.

The [(IPr)Pd(acac)Cl], [(IMes)Pd(acac)Cl] catalysts were prepared according to the literature described in N. Marion et al (Adv. Synth. Catal. 2007, 349, 2380-2384).

The polymeric support loaded with 1-(mesityl)imidazolium (PS-IMes-HCl), was prepared according to the literature described in D.-H. Lee, et al. (Org. Lett. 2008, 10, 1609-1612).

The amount of imidazolium on the imidazolium-loaded polymer support (PS-IMes-HCl) was determined by evaluating the N content by elemental analysis (N 0.89%, i.e. a catalyst amount of: 0.32 mmol/g).

The (PS-IMes-HCl) support is used for the preparation of the [(PS-IMes)Pd(acac)Cl] catalyst described in example 2.

### Example 2—Preparation of [(PS-IMes)Pd(acac)Cl] Catalyst

In a flask fitted with a magnetic bar and a condenser, Pd(acac)₂ (456 mg, 1.5 mmol), an imidazolium-loaded polymer support (PS-IMes-HCl) (2 g, 0.32 mmol/g) and 1,4-dioxane (30 ml) were introduced and the reaction mixture formed was heated at 100° C. for 16 hr. The reaction mixture was then cooled to room temperature, filtered and the polymer support was washed vigorously with distilled water (5×10 ml), methanol (5×10 ml) and dried under reduced pressure to give [(PS-IMes) Pd(acac)Cl] (2.1 g).

The amount of Pd loaded onto the polymer support was determined using ICP-AES analysis. The polymer-supported palladium-N-heterocyclic complex (50 mg) was treated with a mixture (25 ml) of hydrochloric acid and nitric acid (1:1, v/v) at room temperature for 30 min. The orange solution formed was filtered and washed with distilled water. The filtrate and wash solution were combined to determine the amount of Pd by inductively coupled plasma atomic emission spectrometry (ICP-AES). The amount of Pd was calculated to be 0.29 mmol/g of support.

### Example 3: Representative Procedure for Comparative Tests with Pd-Phosphine Catalysts

### Test A:

In a general procedure, a 450 ml Parr autoclave equipped with a stirring bar was charged with palladium (II) acetylacetonate (91.3 mg, 0.3 mmol), triphenylphosphine (236.1 mg, 0.9 mmol), tetrabutylammonium iodide (0.5 g, 1.5 mmol), triethylamine (0.2 ml, 1.5 mmol), acetonitrile (100 ml) and absolute ethanol (50 ml). The reactor was sealed and the mixture purged three times with nitrogen (5 bars), then twice with oxygen (5 bars). The autoclave was then pressurised with 15 bars of oxygen and then 65 bars of carbon monoxide to give a total pressure of 80 bars; and the reaction medium was stirred at 90° C. for 14 h. The autoclave was then allowed to cool to room temperature before being slowly depressurised and purged three times with nitrogen (5 bar). The contents of the reactor were transferred to a round-bottomed flask and the excess ethanol and solvent were removed using a rotary evaporator and the diethyl oxalate DEO (9.3 g) was recovered by vacuum distillation (boiling point=120° C./30-15 mbars).

For reactions with alcohols, the results are given in terms of the mass of oxalate product obtained and described in terms of the number of NCC catalytic cycles as defined above.

### Test B

Test B was carried out under the same operating conditions as Test A, but in the absence of tetrabutylammonium iodide. Traces of diethyl oxalate were observed by quantitative analysis using gas chromatography.

### Test C

Test C was carried out under the same operating conditions as Test A, but in the absence of triethylamine. Traces of diethyl oxalate were observed by quantitative analysis using gas chromatography.

Table 1 shows the operating conditions and results (mass of oxalate and NCC obtained) for tests carried out with a palladium-phosphine catalyst.

TABLE 1
Operating conditions for tests carried out with a palladium-phosphine
catalyst and yield results (mass of oxalate and NCC obtained)

| | n | | | | | | | | m | |
|---|---|---|---|---|---|---|---|---|---|---|
| | (EtOH), | [Pd]cat, | nBu₄NI, | base, | solvent, | O₂/CO, | T, | Time, | (DEO) | |
| Ex | mmol | mol % | mol % | mol % | ml | bar | ° C. | h | g | NCC |
| A | 856 | Pd(acac)₂:PPh₃ = | 0.18 | NEt₃, | CH₃CN, | 15/65 | 90 | 14 | 9.3 | 212 |
| | | 1:30.04 | | 0.18 | 100 | | | | | |
| B | 856 | Pd(acac)₂:PPh₃ = | — | NEt₃, | CH₃CN, | 15/65 | 90 | 14 | traces | — |
| | | 1:30.04 | | 0.18 | 100 | | | | | |
| C | 856 | Pd(acac)₂:PPh₃ = | 0.18 | — | CH₃CN, | 15/65 | 90 | 14 | traces | — |
| | | 1:30.04 | | | 100 | | | | | |

### Example 4: Representative Procedure for the Catalytic Oxidative Carbonylation of Aliphatic Alcohols to Oxalates Using Homogeneous Palladium Catalysts

In a general procedure, a 450 ml Parr autoclave fitted with a stirring bar was charged with homogeneous Pd—NHC complex (0.3 mmol), tetrabutylammonium iodide (0.5 g, 1.5 mmol), triethylamine (0.2 ml, 1.5 mmol), acetonitrile (100 ml) and aliphatic alcohol (50 ml). The reactor was sealed and the mixture purged three times with nitrogen (5 bars), then twice with oxygen (5 bars). The autoclave was then pressurised with 15 bars of oxygen followed by 65 bars of carbon monoxide to bring the total pressure to 80 bars, and the reaction mixture was stirred at 90° C. for 14 h. The autoclave was then allowed to cool to room temperature before being slowly depressurised and purged three times with nitrogen (5 bars). The contents of the reactor were transferred to a round-bottomed flask and the excess aliphatic alcohol and solvent were removed using a rotary evaporator and the dialkyl oxalate (DEO) was recovered by vacuum distillation (boiling point=120° C./50-15 mbars). The reported dialkyl oxalate yield was calculated on the isolated yield by mass and a Catalytic Cycle Number NCC was calculated as defined above.

Table 2 shows the operating conditions and the results (mass of oxalate and NCC obtained) of the tests carried out with a homogeneous Pd—NHC catalyst according to the invention.

TABLE 2
Operating conditions for the tests carried out with a homogeneous Pd-NHC catalyst
and the results for yield (mass of oxalate obtained) and NCC.

| | n | | | base, | | | | | m | |
|---|---|---|---|---|---|---|---|---|---|---|
| | (EtOH), | [Pd]cat, | nBu₄NI, | mol | solvent, | O₂/CO, | T, | time, | (DEO) | |
| Ex | mmol | mol % | mol % | % | ml | bar | ° C. | h | g | NCC |
| 1 | 856 | 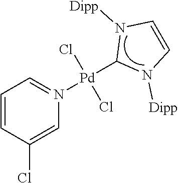 | 0.18 | NEt₃, 0.18 | CH₃CN, 100 | 15/65 | 90 | 14 | 8 | 182 |
| | | 0.04 | | | | | | | | |
| 2 | 856 | 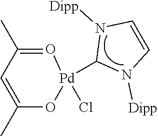 | 0.18 | NEt₃, 0.18 | CH₃CN, 100 | 15/65 | 90 | 14 | 10 | 228 |
| | | 0.04 | | | | | | | | |
| 3 | 856 | 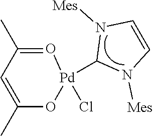 | 0.18 | NEt₃, 0.18 | CH₃CN, 100 | 15/65 | 90 | 14 | 9.5 | 216 |
| | | 0.04 | | | | | | | | |

### Example 5: Influence of Promoter and Base on the Oxidative Carbonylation Reaction of an Alcohol

Test A1—presence of water: Test A1 is carried out under the same operating conditions as test 1 in example 4 but in the presence of water (1 mol %). The reaction yield is equivalent to test 1 in example 4.

Test A2—without base And₃ N: Test A2 is carried out under the same operating conditions as test 1 in example 4 but in the absence of triethylamine. Diethyl oxalate was obtained in an amount of less than 1 g.

Test A3—without nBu₄ NI: Test A3 was carried out under the same operating conditions as test 1 in example 4 but in the absence of tetrabutylammonium iodide. Traces of diethyl oxalate were observed (less than 0.1 g).

Table 3 shows the operating conditions and results (mass of oxalate obtained and NCC) of the tests carried out.

TABLE 3
shows the operating conditions for the tests carried out with Pd-NHC and the results
for yield (mass of oxalate obtained) and NCC.

| | n | | | Et₃N, | | O₂/ | | | m | |
|---|---|---|---|---|---|---|---|---|---|---|
| | (EtOH), | [Pd]cat, | nBu₄NI, | mol | CH₃CN, | CO, | T, | Time, | (DEO), | |
| # | mmol | mol % | mol % | % | ml | Bar | ° C. | h | g | NCC |
| 1 | 856 | 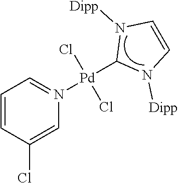 | 0.18 | 0.18 | 100 | 15/65 | 90 | 14 | 8 | 182 |
| | | 0.04 | | | | | | | | |
| A1 | 856 | 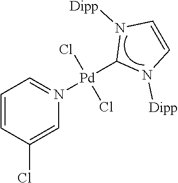 | 0.18 | 0.18 | 100 et H₂O (1 mol %) | 15/65 | 90 | 14 | 7.8 | 178 |
| | | 0.04 | | | | | | | | |
| A2 | 856 | 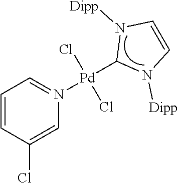 | 0.18 | — | 100 | 15/65 | 90 | 14 | <1.0 | — |
| | | 0.04 | | | | | | | | |
| A3 | 856 | 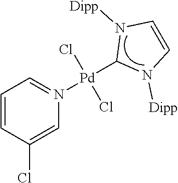 | — | 0.18 | 100 | 15/65 | 90 | 14 | <0.1 | — |
| | | 0.04 | | | | | | | | |

### Example 6: Representative Procedure for the Catalytic Oxidative Carbonylation of Aliphatic Alcohols to Oxalates Using a Heterogeneous Pd—NHC Catalyst According to the Invention

In a general procedure, a 450 ml Parr autoclave fitted with a stirring bar was charged with heterogeneous Pd—NHC complex (1.2 g, 0.29 mmol Pd/g, 0.35 mmol Pd), tetrabutylammonium iodide (0.5 g, 1.5 mmol), triethylamine (0.2 ml, 1.5 mmol), acetonitrile (100 ml) and aliphatic alcohol (50 ml). The reactor was sealed and the reaction mixture was purged three times with nitrogen (5 bars), then twice with oxygen (5 bars). The autoclave was then pressurised with 15 bar of oxygen and then 65 bar of carbon monoxide to bring the total pressure to 80 bar; and the reaction mixture was stirred at 90° C. for 14 h. The autoclave was then allowed to cool to room temperature before being slowly depressurised and purged three times with nitrogen (5 bar). The contents of the reactor were filtered to recover the catalyst. The filtrate was transferred to a round-bottomed flask and the excess aliphatic alcohol and solvent were removed using a rotary evaporator and the dialkyl oxalate was recovered by vacuum distillation (boiling point=120° C./50-15 mbars).

The reported dialkyl oxalate yield was calculated on the isolated yield by mass and a Catalytic Cycle Number NCC was calculated as defined above.

Table 4 shows the operating conditions and the results (mass of oxalate and NCC obtained) of a test carried out with a heterogeneous Pd—NHC catalyst according to the invention.

TABLE 4
Operating conditions for the test with the [(PS-IMes)Pd(acac)Cl] catalyst and yield
results (mass of oxalate obtained) and NCC.

| | n | | | base, | | O₂/ | | | m | |
|---|---|---|---|---|---|---|---|---|---|---|
| | (EtOH), | | nBu₄NI, | mol | solvent, | CO, | T, | Time, | (DEO) | |
| # | mmol | [Pd]cat, mol % | mol % | % | ml | bar | ° C. | h | g | NCC |
| 4 | 856 | 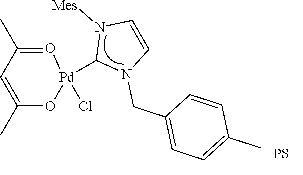 | 0.18 | NEt₃, 0.18 | CH₃CN, 100 | 15/65 | 90 | 14 | 8 | 182 |
| | | 0.04 | | | | | | | | |

### Example 7: Representative Procedure for the Recyclability Study of the [(PS-IMes)Pd(Acac)Cl] Complex

The reaction was carried out as mentioned in Example 6 above in a typical experimental procedure. However, once the reaction was complete, vented and purged with nitrogen, the catalyst was filtered and washed vigorously with distilled water (5×10 ml) and methanol (5×10 ml) to remove any traces of product or reagents present. The filtered catalyst was then dried under reduced pressure before further recycling. The dried catalyst was then used in a catalyst recyclability experiment, and it was found that the recovered catalyst could be reused for at least five consecutive cycles giving a good to appreciable yield of the desired product.

The recyclability study shows that the heterogeneous catalyst can be used in a continuous flow reactor.

Table 5 shows the conditions and results of the recyclability tests on the [(PS-IMes)Pd(acac)Cl]complex.

TABLE 5
Recyclability test conditions for the [(PS-IMes)Pd(acac)Cl] complex and performance
results.

| | | | | m | | |
|---|---|---|---|---|---|---|
| | Cycle | n[Pd], | | (DEO), | | NCC |
| exp | N | mmol | Carbonylation conditions | g | NCC | total |
| C1 | 1 | 0.3 | 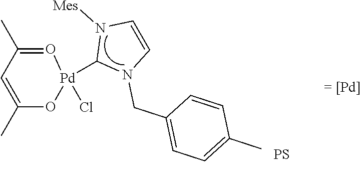 | 8 | 182 | 1096 |
| C2 | 2 | 0.24 | 856 mmol (50 mL) EtOH; 1.5 mmol NEt₃; | 6 | 171 | |
| C3 | 3 | 0.198 | 1.5 mmol nBu₄NI; CH₃CN 100 mL; | 6 | 207 | |
| C4 | 4 | 0.15 | CO/O₂ = 65/15, 90° C., 14h | 6 | 274 | |
| C5 | 5 | 0.1125 | | 4.3 | 262 | |

The results of these manual recycling experiments show that the Pd—NHC catalyst is stable. The recovered catalyst is not, or only slightly, sensitive to water and oxygen. In addition, there is no loss of efficiency in terms of NCC during the recycling of the catalyst as part of several successive catalytic reactions.

By switching to flow-through, manual recycling is no longer necessary, preventing catalyst loss during washings, so efficiency should be maintained during the reaction as reaction conditions will be stable.

### Oxamide

### Example 8: Representative Procedure for the Catalytic Oxidative Carbonylation of Amines to Oxamides

PEPPSI™—IPr, tetrabutylammonium iodide, base, solvent and piperidine were introduced into a 450 ml Parr autoclave fitted with a stir bar. The reactor was sealed, the mixture purged three times with nitrogen (5 bar) and then twice with oxygen (5 bars). The autoclave was then pressurised with 10 bar of oxygen, followed by 65 bars of carbon monoxide to give a total pressure of 75 bar, and the reaction medium was stirred at room temperature for 18 hours. The pressure was then carefully released and the autoclave was purged three times with nitrogen (5 bar). The contents of the reactor were transferred to a round-bottomed flask and the volatile substances were removed under reduced pressure. The residue was then extracted in toluene, filtered over silica gel (2-3 cm) and the solution evaporated to dryness to give 1, 1′-oxalyl dipiperidine in the form of an off-white powder.

The oxamide yield is given by mass of isolated product and a Catalytic Cycle Number (CCN) has been calculated as defined above.

### Example 9: Influence of Solvent on the Catalytic Oxidative Carbonylation of Amine to Oxamide

### M1-THF Test

Into a 450 ml Parr autoclave fitted with a stirring bar were introduced PEPPSI™_JPr Catalyst (62.5 mg, 0.092 mmol), tetrabutylammonium iodide (2.12 g, 5.75 mmol), potassium carbonate (1.59 g, 11.5 mmol), THF (200 ml) and piperidine (22.7 ml, 230 mmol). The reactor was sealed and the mixture purged three times with nitrogen (5 bars), then twice with oxygen (5 bar). The autoclave was then pressurised with 10 bar of oxygen and then 65 bar of carbon monoxide to give a total pressure of 75 bar; and the reaction medium was stirred at room temperature for 18 h. After this time, the pressure was carefully released and the autoclave was purged three times with nitrogen (5 bar). The contents of the reactor were transferred to a round-bottomed flask and the volatile substances were removed under reduced pressure. The residue was then extracted in toluene, filtered over silica gel (2-3 cm) and the solution evaporated to dryness to give 1, 1-oxalyl dipiperidine as an off-white powder (9.5 g; 42 mmol).

### Test M2—Acetonitrile

Test M2 was carried out under the same conditions as test M1, except that 200 mL of acetonitrile was used instead of THF.

Result: 9.5 g of oxamide 2 were isolated.

### Test M3—Solvent-Free

Test M3 was carried out under the same conditions as test M1, in the absence of solvent.

Result: 4.2 g of oxamide 2 were isolated.

Table 6 shows the conditions relating to the nature and presence of the solvent and the results of catalytic oxidative carbonylation tests on amines to oxamides using homogeneous Pd—NHC catalysts according to the invention.

TABLE 6
Operating conditions with different solvents for tests with piperidine using Pd-NHC
catalysts and yield results.

| | n | | | | | O₂/ | | | |
|---|---|---|---|---|---|---|---|---|---|
| | (piperidine), | [Pd]cat, | nBu₄NI, | base, | solvent, | CO, | T, | Time, | Yield |
| Ex | mmol | mol % | mol % | mol % | ml | bar | ° C. | h | (oxamide) |
| M1 | 230 | 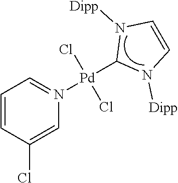 | 2.5 | K2CO₃ 5 | THF, 200 | 10/65 | 25 | 18 | 9.5 g NCC = 460 |
| | | 0.04 | | | | | | | |
| M2 | 230 | 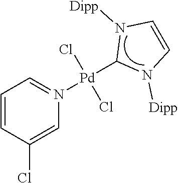 | 2.5 | K2CO₃ 5 | CH₃CN, 200 | 10/65 | 25 | 18 | 9.5 g NCC = 460 |
| | | 0.04 | | | | | | | |
| M3 | 230 | 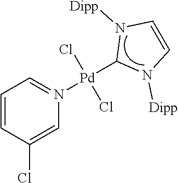 | 2.5 | K2CO₃ 5 | — | 10/65 | 25 | 18 | 4.2 g NCC = 203 |
| | | 0.04 | | | | | | | |

The reaction takes place in the presence of both THE and CH₃CN with the same efficiency, as demonstrated in tests M1 and M2.

The reaction takes place in the absence of solvent, as demonstrated by test M3.

### Example 10: Influence of the Base on the Catalytic Oxidative Carbonylation of Amine to Oxamide

### M4 Test

Test M4 was carried out under the same conditions as test M1, except that the base used was triethylamine (5 mol %).

### M5 Test

Test M5 was carried out under the same conditions as test M1, with no base added.

Table 7 reports the conditions on the nature and presence of the added base and the results of catalytic oxidative carbonylation tests of amines to oxamides with homogeneous Pd—NHC catalysts according to the invention.

TABLE 7
Operating conditions with different bases for tests with piperidine using Pd-NHC
catalysts and yield results.

| | n | | | | | O₂/ | | | |
|---|---|---|---|---|---|---|---|---|---|
| | (piperidine), | [Pd]cat, | nBu₄NI, | base, | solvent, | CO, | T, | Time, | Yield |
| # | mmol | mol % | mol % | mol % | ml | bar | ° C. | h | (oxamide) |
| M1 | 230 | 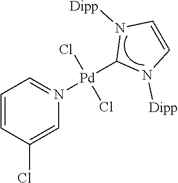 | 2.5 | K2CO₃ 5 | THF, 200 | 10/65 | 25 | 18 | 9.5 g NCC = 460 |
| | | 0.04 | | | | | | | |
| M4 | 230 | 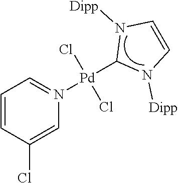 | 2.5 | NEt₃ 5 | THF, 200 | 10/65 | 25 | 18 | 9.3 g NCC = 450 |
| | | 0.04 | | | | | | | |
| M5 | 230 | 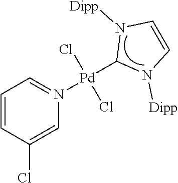 | 2.5 | — | THF, 200 | 10/65 | 25 | 18 | 17.0 g NCC = 823 |
| | | 0.04 | | | | | | | |

Organic and inorganic bases have comparable effects on the reaction, as demonstrated by the results of M1 and M4.

The reaction is more efficient in the absence of a base added to the reaction mixture, as demonstrated in test M5.

### Example 11: Use of [(PS-IMes)Pd(Acac)Cl] Catalyst for the Catalytic Oxidative Carbonylation of Amines to Oxamides Using a Heterogeneous Pd—NHC Catalyst

### Test M6

In a 450 ml Parr autoclave fitted with a stirring bar, [(PS-Imes)Pd(acac)Cl] catalyst (317 mg, 0.29 mmol Pd/g, 0.092 mmol Pd), tetrabutylammonium iodide (2.12 g, 5.75 mmol), THE (200 ml) and piperidine (22.7 ml, 230 mmol) were introduced. The reactor was sealed and the mixture purged three times with nitrogen (5 bars), then twice with oxygen (5 bar). The autoclave was then pressurised with 10 bars of oxygen followed by 65 bar of carbon monoxide to give a total pressure of 75 bar, and the reaction medium was stirred at room temperature for 18 hours. The pressure was then carefully released and the autoclave was purged three times with nitrogen (5 bars). The contents of the reactor were filtered to recover the catalyst. The filtrate was transferred to a round-bottomed flask and the volatiles were removed under reduced pressure. The residue was then extracted in toluene, filtered over silica gel (2-3 cm) and the solution evaporated to dryness to give 1, 1′-oxalyl dipiperidine in the form of an off-white powder.

Table 8 shows the operating conditions for a test with a homogeneous catalyst and a test with a supported catalyst for the catalytic oxidative carbonylation of the amines piperidine to the oxamide 1,1′-oxalyl dipiperidine, and the yield results.

TABLE 8
Operating conditions with a homogeneous catalyst and with a supported catalyst for
oxidative carbonylation with piperidine and yield results.

| | n | | | base, | | O₂/ | | | |
|---|---|---|---|---|---|---|---|---|---|
| | (piperidine), | | nBu₄NI, | mol | solvent, | CO, | T, | Time, | Yield |
| Ex | mmol | [Pd]cat, mol % | mol % | % | ml | bar | ° C. | h | (oxamide) |
| M5 | 230 | 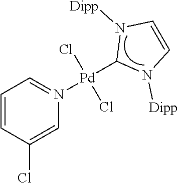 | 2.5 | — | THF, 200 | 10/65 | 25 | 18 | 17.0 g NCC = 823 66% |
| | | 0.04 | | | | | | | |
| M6 | 230 | 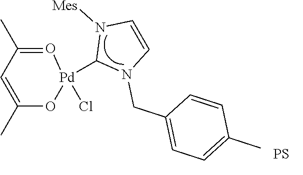 | 2.5 | — | THF, 200 | 10/65 | 25 | 18 | 18.0 g NCC = 872 70% |
| | | 0.04 | | | | | | | |

### Example 12: Influence of the Introduction of CO and O₂

### M7 Test

Test M7 was carried out under the same conditions as test M5, except that after 18 hours of stirring at room temperature, the reactor was purged and pressurised a second time with 10 bars of oxygen and 65 bars of CO; and the reaction medium was stirred at room temperature for a further 18 hours.

Table 9 shows the operating conditions.

TABLE 9
Operating conditions on the influence of pressure and time conditions.

| | n | | | base, | | O₂/ | | | |
|---|---|---|---|---|---|---|---|---|---|
| | (piperidine), | | nBu₄NI, | mol | solvent, | CO, | T, | Time, | Yield |
| Ex | mmol | [Pd]cat, mol % | mol % | % | ml | bar | ° C. | H | (oxamide) |
| M5 | 230 | 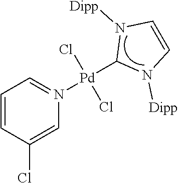 | 2.5 | — | THF, 200 | 10/65 | 25 | 18 | 17.0 g NCC = 823 66% |
| | | 0.04 | | | | | | | |
| M7 | 230 | 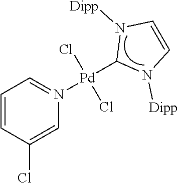 | 2.5 | — | THF, 200 | 10/65 for 18h + 10/65 for 18h | 25 | 36 | 25.0 g NCC = 1211 97% |
| | | 0.04 | | | | | | | |

Test M7 shows that re-pressurising the reactor with additional CO and O₂ during the reaction enables full yield to be achieved.

### Example 13: Representative Procedure for the Catalytic Oxidative Carbonylation of Aliphatic Alcohols to Oxalates Using Pt—NHC Catalysts

In a general procedure, a 450 ml Parr autoclave fitted with a stirring bar is charged with homogeneous Pt—NHC complex (0.6 mmol), tetrabutylammonium iodide (1.1 g, 3.0 mmol), triethylamine (0.4 ml, 3.0 mmol), acetonitrile (100 ml) and aliphatic alcohol (50 ml). The reactor was sealed and the mixture purged three times with nitrogen (5 bar), then twice with oxygen (5 bar). The autoclave was then pressurised with 15 bar of oxygen and then 65 bar of carbon monoxide to bring the total pressure to 80 bar, and the reaction mixture was stirred at 90° C. for 14 hours. The autoclave was then allowed to cool to room temperature before being slowly depressurised and purged three times with nitrogen (5 bars). The contents of the reactor were transferred to a round-bottomed flask and the excess aliphatic alcohol and solvent were removed using a rotary evaporator. The dialkyl oxalate (DEO) was recovered by vacuum distillation (boiling point=120° C./50-15 mbar). The yield of dialkyl oxalate reported is calculated on the isolated yield by mass and a Catalytic Cycle Number NCC has been calculated as defined above.

### Example 14: Representative Procedure for the Catalytic Oxidative Carbonylation of Amines to Oxamides Using Pt—NHC Catalysts

Pt—NHC catalyst (1.0 mmol), tetrabutylammonium iodide (2.12 g, 5.75 mmol), THE (200 ml) and piperidine (22.7 ml, 230 mmol) were added to a 450 ml Parr autoclave fitted with a stirring bar. The reactor was sealed and the mixture purged three times with nitrogen (5 bar), then twice with oxygen (5 bar). The autoclave was then pressurised with 10 bar of oxygen followed by 65 bar of carbon monoxide to give a total pressure of 75 bar, and the reaction medium was stirred at 90° C. for 14 h. The autoclave was then allowed to cool to room temperature before being slowly depressurised and purged three times with nitrogen (5 bar). The contents of the reactor were transferred to a round-bottomed flask and the volatile substances were removed under reduced pressure. The residue was then extracted in toluene, filtered through silica gel (2-3 cm) and the solution evaporated to dryness to give 1,1′-oxalyl dipiperidine.

### Example 15: Synthesis of a Pd—NHC Catalyst Supported on Silica Gel (PdCl NHC/Si₂) of the Following Formula

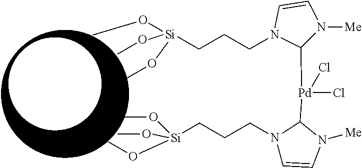

The catalyst was prepared in the following steps:

### A) Synthesis of 1-methyl-3-(trimethoxysilylpropyl)-imidazolium chloride according to the following reaction scheme

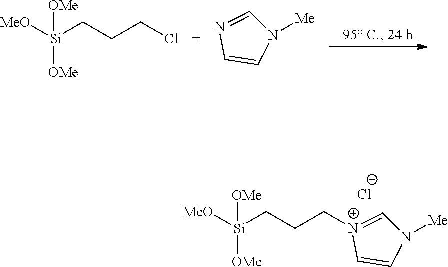 [SMILES: CO[Si](CCCCl)(OC)OC ; Cn1ccnc1 ; [Cl-] ; CO[Si](CCC[n+]1ccn(C)c1)(OC)OC]

In a flask fitted with a magnetic bar and a condenser, the mixture of N-methyl imidazole (freshly distilled) (10.3160 g, 0.1258 mol) and 3-chloropropyl trimethoxysilane (25 g, 0.1258 mol) was refluxed at 95° C. for 24 hours. After cooling to room temperature, the reaction mixture was washed with diethyl ether and dried in vacuo to give the desired product.

### B) Immobilisation of 1-methyl-3-(trimethoxysilylpropyl)-imidazolium chloride on the surface of silica gel according to the following reaction scheme

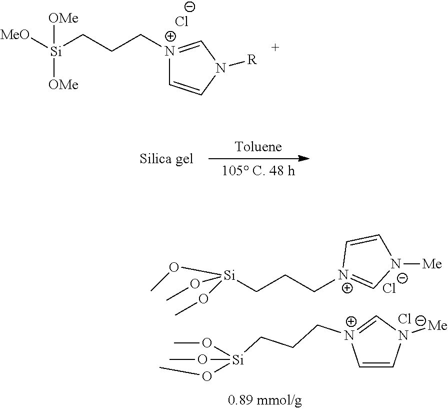

To a solution of 1-methyl-3-(trimethoxysilylpropyl)-imidazolium chloride (0.70 g, 2.2 mmol) in toluene silica gel was added. The mixture was stirred at 105° C. for 12 hours. After cooling, the reaction mixture was filtered and washed with CH₂Cl₂ (3*10 mL), and dried at 60° C. in vacuo to give the silica-supported ionic liquid (2.39 g). Elemental analysis showed the presence of 0.89 mmol of ligand on 1.0 g of support.

### C) Preparation of an NHC—Pd Complex Supported on Silica Gel According to the Following Reaction Scheme

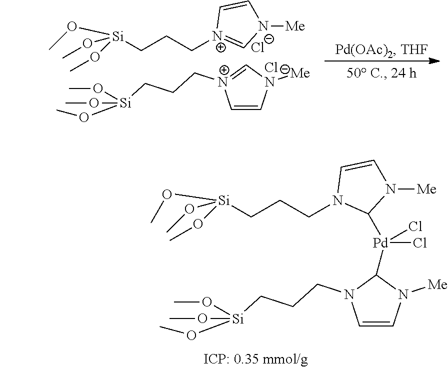

To a solution of ligand supported on silica (1.0 g, 0.89 mmol) in THE (5 mL) was added Pd(OAc)₂ (101 mg, 0.45 mmol). The mixture was stirred for 4 h at 60° C. and then for a further 30 min at 100° C. The NHC—Pd complex supported on silica was filtered through a sinter and washed with water and then with CH₂Cl₂ (3×10 mL). Once washed, the catalyst was dried.

ICP analysis showed the presence of 0.35 mmol of Pd on 1 g of support.

With a quantity of 0.35 mmol of Pd on 1 g of support, better complexation of palladium on this supported ligand is observed compared with the [(PS-IMes)Pd(acac)Cl] catalyst of example 2, which comprises a quantity of 0.29 mmol/g.

### Example 16: Recyclability Study of the PdCl Catalyst NHC/Si₂

### Protocol

In a general procedure, a 450 ml Parr autoclave fitted with a stirring bar was charged with heterogeneous Pd—NHC complex (1.0 g, 0.35 mmol Pd/g), tetrabutylammonium iodide (0.5 g, 1.5 mmol), triethylamine (0.2 ml, 1.5 mmol), acetonitrile (100 ml) and aliphatic alcohol (50 ml). The reactor was sealed and the reaction mixture purged three times with nitrogen (5 bars), then twice with oxygen (5 bar). The autoclave was then pressurised with 15 bar of oxygen followed by 65 bars of carbon monoxide to bring the total pressure to 80 bar, and the reaction mixture was stirred at 90° C. for 14 h. The autoclave was then allowed to cool to room temperature before being slowly depressurised and purged three times with nitrogen (5 bar). The contents of the reactor were filtered to recover the catalyst. The filtrate was transferred to a round-bottomed flask and the excess aliphatic alcohol and solvent were removed using a rotary evaporator and the dialkyl oxalate was recovered by vacuum distillation (boiling point=120° C./50-15 mbars). The reported dialkyl oxalate yield was calculated on the isolated yield by mass and a Catalytic Cycle Number NCC was calculated as defined above.

Table 10 shows the conditions and results of recyclability tests with the PdCl₂NHC/Si catalyst.

TABLE 10
Recyclability test conditions with the PdCl₂ NHC/Si catalyst and the yield and NCC
results.

| | | n[Pd], | | m(DEO), | | NCC |
|---|---|---|---|---|---|---|
| Exp | Cycle | mmol | Carbonylation condition | g | NCC | total |
| A | 1 | 0.35 | 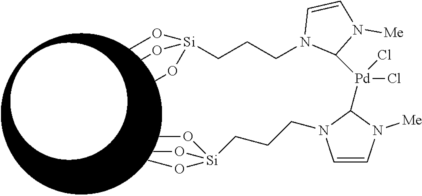 | 11 | 215 | 561 |
| B | 2 | 0.3 | 856 mmol (50 mL) EtOH; 1.5 | 8 | 182 | |
| C | 3 | 0.25 | mmol Net₃; 1.5 mmol nBu₄NI; | 6 | 164 | |
| | | | CH₃CN 100 mL; CO/O₂ = 65/15, | | | |
| | | | 90° C., 14h | | | |

### Example 17: Preparation of the Palladium Catalyst with an NHC Group Supported with Iodinated Ligands, PdI₂ NHC/PS of the Following Formula

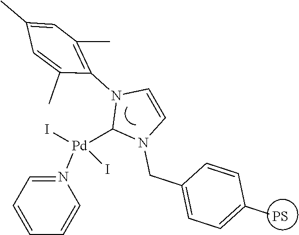

In a flask fitted with a magnetic bar and a condenser, PdCl₂ (266 mg, 1.5 mmol), an imidazolium-loaded polymeric support (PS-IMes-HCl) (1 g, 1.6 mmol/g), potassium iodide (1.2 g, 7.5 mmol), potassium carbonate (1.03 g, 7.5 mmol) and pyridine (7 ml) were introduced, potassium iodide (1.2 g, 7.5 mmol), potassium carbonate (1.03 g, 7.5 mmol) and pyridine (7 ml) were introduced and the reaction mixture formed was heated at 80° C. for 16 h. The reaction mixture was then cooled to room temperature, filtered and the polymer support was washed vigorously with distilled water (5×10 ml), methanol (5×10 ml) and dried under reduced pressure to give (1.6 g) of the desired product.

The amount of Pd loaded onto the polymer support was determined using ICP-AES analysis. The polymer-supported palladium-N-heterocyclic complex (50 mg) was treated with a mixture (25 ml) of hydrochloric acid and nitric acid (1:1, v/v) at room temperature for 30 min. The orange solution formed was filtered and washed with distilled water. The filtrate and wash solution were combined to determine the amount of Pd by inductively coupled plasma atomic emission spectrometry (ICP-AES). The amount of Pd was calculated to be 0.9 mmol/g of support.

### Example 18: Carbonylation Procedure without Added Iodine Salt

The Inventors have succeeded in dispensing with the addition of iodine salt. The iodine is incorporated directly into the catalyst, such as the PdI₂ NHC/PS prepared in example 17, making it possible to maintain efficient catalyst reoxidation while avoiding the need to add additional iodine salt.

### Protocol

A 450 ml Parr autoclave fitted with a stirring bar was charged with heterogeneous Pd—NHC complex (1.0 g, 0.35 mmol Pd/g), triethylamine (0.2 ml, 1.5 mmol), acetonitrile (100 ml) and ethanol (50 ml). The reactor was sealed and the reaction mixture purged three times with nitrogen (5 bar), then twice with oxygen (5 bars). The autoclave was then pressurised with 15 bars of oxygen followed by 65 bar of carbon monoxide to bring the total pressure to 80 bar, and the reaction mixture was stirred at 90° C. for 14 h. The autoclave was cooled to room temperature before being slowly depressurised and purged three times with nitrogen (5 bars). The contents of the reactor were filtered to recover the catalyst. The filtrate was transferred to a round-bottomed flask and the residual ethanol and acetonitrile were removed using a rotary evaporator. The purified dialkyl oxalate was recovered by vacuum distillation (boiling point=120° C./50-15 mbar). The reported dialkyl oxalate yield was calculated on the isolated yield by mass and a Catalytic Cycle Number NCC was calculated as defined above.

Table 11 shows the operating conditions and the results obtained.

TABLE 11
Operating conditions for carbonylation using the PdI₂ NHC/PS catalyst without
adding an iodine salt as a promoter, and the yield and NCC results.

| | | | | | CO | O₂ | Time, | Quantity | |
|---|---|---|---|---|---|---|---|---|---|
| Reaction | Cat | Ethanol | MeCN | T, (° C.) | (Bar) | (Bar) | (h) | (g) | NCC |
| 1 | PdI | 50 mL | 100 mL | 90 | 65 | 15 | 16 | 8.5 g | 64 |
| | NHC/PS₂ | | | | | | | | |
| | 0.9 mmol | | | | | | | | |
| | (1 g) | | | | | | | | |

### Example 19: Representative Procedure for the Catalytic Oxidative Carbonylation of Amines to Oxamides without Added Iodine Salt

A 450 ml Parr autoclave fitted with a stirring bar was charged with the heterogeneous PdI₂ NHC/PS complex prepared according to example 17 (1.0 g, 0.9 mmol Pd/g), THE (200 ml) and piperidine (22.7 ml, 230 mmol). The reactor was sealed and the mixture purged three times with nitrogen (5 bars), then twice with oxygen (5 bars). The autoclave was then pressurised with 10 bar of oxygen followed by 65 bar of carbon monoxide to give a total pressure of 75 bar, and the reaction medium was stirred at room temperature for 18 hours. The pressure was then carefully released and the autoclave was purged three times with nitrogen (5 bars). The contents of the reactor were filtered to recover the catalyst. The filtrate was transferred to a round-bottomed flask and the volatiles were removed under reduced pressure. The residue was then extracted in toluene, filtered over silica gel (2-3 cm) and the solution evaporated to dryness to give 1, 1′-oxalyl dipiperidine in the form of an off-white powder.

1-17. (canceled)

18. A method for selectively preparing an oxalate compound or an oxamide compound comprising:
a step A of contacting an alcohol or an amine, respectively, with:
carbon monoxide, in particular used from 1.0 to 10.0 MPa, in particular 6.5 MPa,
an oxidant, in particular oxygen or air, preferably oxygen used at 0.5 to 2.5 MPa,
an M-NHC catalyst, wherein M represents Pd or Pt and NHC represents an N-heterocyclic carbene group, comprising at least one M atom linked to at least one N-heterocyclic carbene ligand,
optionally a promoter,
optionally a base,
optionally a solvent,
to obtain a reaction medium,
to obtain the oxalate compound or the oxamide compound.

19. The method for selectively preparing an oxalate compound or an oxamide compound according to claim 18, comprising:
a step A of contacting an alcohol or an amine, respectively, with a promoter.

20. The method for selectively preparing an oxalate compound or an oxamide compound according to claim 18, comprising:
a step A of contacting an alcohol or an amine, respectively, with a base.

21. The method for selectively preparing an oxalate compound or an oxamide compound according to claim 18, comprising:
a step A of contacting an alcohol or an amine, respectively, with a solvent.

22. The method for selectively preparing an oxalate compound or an oxamide compound according to claim 18, comprising a step B of heating the reaction medium.

23. The method for selectively preparing an oxalate compound or an oxamide compound according to claim 19, wherein the promoter is selected from: tetrabutylammonium iodide (Bu₄NI), sodium iodide (NaI) and potassium iodide (KI), preferably tetrabutylammonium iodide.

24. The method for selectively preparing an oxalate compound or an oxamide compound according to claim 20, wherein the base is selected from potassium carbonate (K₂CO₃), sodium carbonate (Na₂CO₃), potassium tert-butylate (KotBu), potassium phosphate (K₃PO₄) and triethylamine (Et₃N).

25. The method for selectively preparing an oxalate compound or an oxamide compound according to claim 21, wherein the solvent is selected from acetonitrile, toluene, 1,4-dioxane, tetrahydrofuran, ethanol, methanol and ethyl acetate, preferably acetonitrile and tetrahydrofuran.

26. The method for selectively preparing an oxalate compound or an oxamide compound according to claim 18, wherein the catalyst corresponds to Formula II: 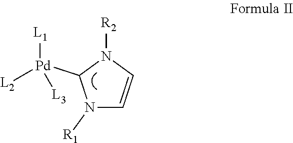
wherein:
R₁ and R₂ independently of one another represent a group chosen from:
C₁ to C₁₀ linear or branched alkyl
C₃ to C₁₀ cycloalkyl
C₆ to C₂₀ aryl or C₃ to C₂₀ heteroaryl, and
C₇ to C₂₀ alkyl-aryl or C₄ to C₂₀ alkyl-heteroaryl,
L₁ represents a halogen atom chosen from Cl, Br and I, and
or L₂ and L₃ represent a bidentate ligand, in particular chosen from acetylacetonate (acac), allyl, cinnamyl and acetate, preferably acetylacetonate,
or L₂ represents a halogen atom chosen from Cl, Br and I,
and L₃ represents a monodentate ligand, in particular selected from: pyridine, 3-chloropyridine, acetonitrile, triethylamine, or a phosphine-based ligand, in particular triphenylphosphine, preferably 3-chloropyridine,
said compound of Formula II can be bonded to a support, in particular a polymer or silica, by at least one of the groups R₁ or R₂.

27. The method for selectively preparing an oxalate compound or an oxamide compound according to claim 26, wherein the catalyst corresponds to Formula II, wherein:
L₁ represents an iodine atom, and
or L₂ and L₃ are linked and together represent a bidentate ligand,
or L₂ represents a halogen atom chosen from Cl, Br and I and L₃ represents a monodentate ligand.

28. The method for selectively preparing an oxalate compound or an oxamide compound according to claim 26, wherein the catalyst corresponds to Formula II, wherein:
L₁ and either L₂ each represent an iodine atom
and L₃ represents a monodentate ligand

29. The method for selectively preparing an oxalate compound or an oxamide compound according to claim 18, wherein the catalyst corresponds to Formula III: 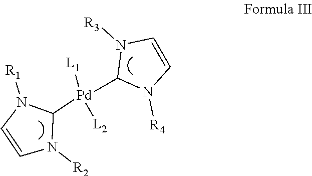
wherein:
R₁, R₂, R₃ and R₄ independently of one another represent a group selected from:
C₁ to C₁₀ linear or branched alkyl
C₃ to C₁₀ cycloalkyl
C₆ to C₂₀ aryl or C₃ to C₂₀ heteroaryl, and
C₇ to C₂₀ alkyl-aryl or C₄ to C₂₀ alkyl-heteroaryl,
or L₁ and L₂ represent each other independently:
a halogen atom selected from Cl, Br and I, or
a monodentate ligand,
or L₁ and L₂ are linked and represent a bidentate ligand,
said compound of Formula II can be bonded to a support in particular a polymer or silica, by at least one of the groups R₁, R₂, R₃ or R₄,
optionally one of the groups R₁ or R₂ can be linked to R₃ or R₄ and together represent a bidentate group comprising two N-heterocyclic carbene groups.

30. The method for selectively preparing an oxalate compound or an oxamide compound according to claim 29, wherein the catalyst corresponds to Formula III,
wherein at least one of the groups L₁ and L₂ represents an iodine atom, or
wherein the groups L₁ and L₂ each represent an iodine atom.

31. The method for selectively preparing an oxalate compound or an oxamide compound according to claim 18, wherein the catalyst corresponds to Formula IV: 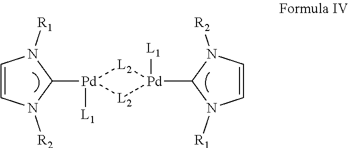
wherein:
R₁ and R₂ independently of one another represent a group chosen from:
C₁ to C₁₀ linear or branched alkyl
C₃ to C₁₀ cycloalkyl
C₆ to C₂₀ aryl or C₃ to C₂₀ heteroaryl, and
C₇ to C₂₀ alkyl-aryl or C₄ to C₂₀ alkyl-heteroaryl,
L₁ and L₂ represent a halogen atom chosen from Cl, Br and I.

32. The method for selectively preparing an oxalate compound or an oxamide compound according to claim 26, wherein the catalyst corresponds to Formula II-1 or Formula II-2 or Formula II-3: 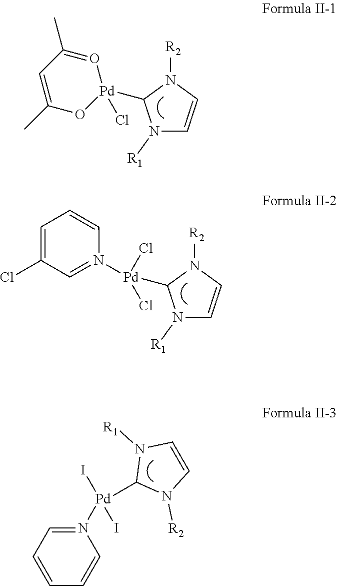
wherein:
R₁ and R₂ independently of one another represent a group chosen from:
C₁ to C₁₀ linear or branched alkyl
C₃ to C₁₀ cycloalkyl
C₆ to C₂₀ aryl or C₃ to C₂₀ heteroaryl, and
C₇ to C₂₀ alkyl-aryl or C₄ to C₂₀ alkyl-heteroaryl,
said catalyst can be bonded to a support, in particular a polymer or silica, by at least one of the groups R₁ or R₂,
in particular said catalyst is selected from: 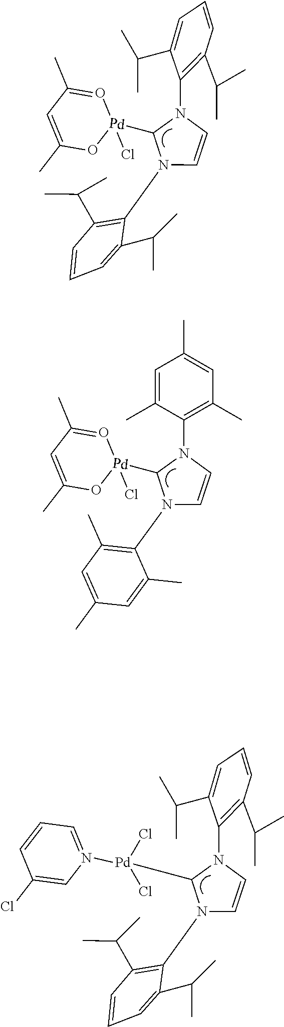
or wherein the catalyst, bound to a support (PS), is chosen from:

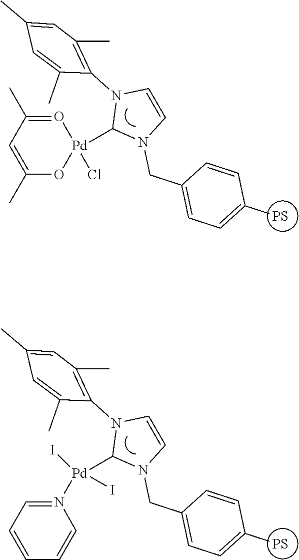

33. The method for selectively preparing an oxalate compound or an oxamide compound according to claim 29, wherein the catalyst corresponds to Formula III-1 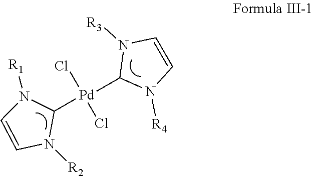
wherein:
R₁, R₂, R₃ and R₄ independently of one another represent a group selected from:
C₁ to C₁₀ linear or branched alkyl
C₃ to C₁₀ cycloalkyl
C₆ to C₂₀ aryl or C₃ to C₂₀ heteroaryl, and
C₇ to C₂₀ alkyl-aryl or C₄ to C₂₀ alkyl-heteroaryl,
said compound of Formula III-1 can be bonded to a support, in particular a polymer or silica, by at least one of the groups R₁, R₂, R₃ or R₄,
optionally one of the groups R₁ or R₂ can be linked to R₃ or R₄ and together represent a bidentate group comprising two N-heterocyclic carbene groups,
in particular said supported catalyst of Formula III-1 is selected from

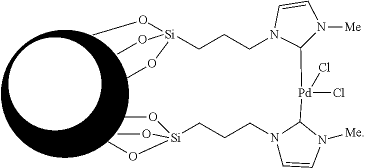

34. The method for selectively preparing an oxalate compound or an oxamide compound according to claim 18, wherein an oxalate compound is selectively prepared and the method comprises:
a step A of contacting an alcohol with
carbon monoxide,
oxygen or air,
a Pd—NHC catalyst, and
optionally a promoter,
optionally a base,
optionally a solvent,
to obtain a reaction medium,
a step B of heating said reaction medium to obtain the oxalate compound, in particular at a temperature of from 25 to 200° C., in particular from 60 to 110° C., preferably about 90° C.

35. The method for selectively preparing an oxalate compound or an oxamide compound according to claim 18, wherein an oxalate compound of Formula 2 is selectively prepared, and wherein step A comprises contacting an alcohol of Formula 1 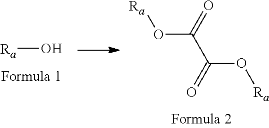
wherein Rₐ represents a group chosen from:
C₁ to C₁₀ linear or branched alkyl
C₃ to C₁₀ cycloalkyl
in particular the alcohol is chosen from methanol, ethanol and isopropanol.

36. The method for selectively preparing an oxalate compound or an oxamide compound according to claim 18, wherein an oxamide compound is selectively prepared, and wherein the method comprises:
a step A of contacting an amine with:
carbon monoxide,
oxygen or air,
optionally a promoter,
a Pd—NHC type catalyst,
a solvent, and
optionally a base, in particular selected from K₂CO₃ or Et₃N,
to obtain a reaction medium comprising the oxamide.

37. The method for selectively preparing an oxalate compound or an oxamide compound according to claim 18, wherein an oxamide compound of Formula 4 is selectively prepared, and wherein step A comprises contacting an amine of Formula 3: 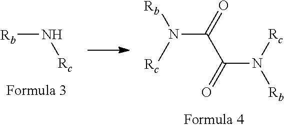
wherein R_{b} and R_{c} independently of one another represent:
a hydrogen atom,
C₁ to C₂₀ linear or branched alkyl group
C₂ to C₂₀ linear or branched alkenyl group
C₁ to C₂₀ linear or branched heteroalkyl group, the heteroatom being in particular O or N,
at least one of the groups R_{b} or R_{c} being other than hydrogen,
R_{b} and R_{c} can form a cycle,
in particular the amine is chosen from: diethylamine, piperidine, pyrrolidine and morpholine.
